SEQUENCE LISTING (131 sequences)

SEQ ID NO: 1 - DNA, 30 bp, Artificial Sequence
  Synthetic Primer
cccggatccctcgtcggcgccgggcccggc

SEQ ID NO: 2 - DNA, 33 bp, Artificial Sequence
  Synthetic Primer
ccctctagagcggacgcacgtggtgcgcgtcca

SEQ ID NO: 3 - DNA, 20 bp, Artificial Sequence
  Synthetic Primer
gtggcactcgtggtgatctt

SEQ ID NO: 4 - DNA, 19 bp, Artificial Sequence
  Synthetic Primer
gagcgaggaagcggaagag

SEQ ID NO: 5 - DNA, 24 bp, Artificial Sequence
  Synthetic Primer
ccccggcgcctacaaggacatcga

SEQ ID NO: 6 - DNA, 24 bp, Artificial Sequence
  Synthetic Primer
ccgccctcctgttgatgttctggt

SEQ ID NO: 7 - DNA, 31 bp, Artificial Sequence
  Synthetic Primer
cccggatcctcagacgccgcggcgtccaccc

SEQ ID NO: 8 - DNA, 49 bp, Artificial Sequence
  Synthetic Primer
cgtggggccgtcgtagtgggacgatgcatgtgagcgactggcgacgaga

SEQ ID NO: 9 - DNA, 49 bp, Artificial Sequence
  Synthetic Primer
tctcgtcgccagtcgctcacatgcatcgtcccactacgacggccccacg

SEQ ID NO: 10 - DNA, 51 bp, Artificial Sequence
  Synthetic Primer
gccgggcaacagtgtggtggtcatgacgcacgatcccgagaaaggaatgcc

SEQ ID NO: 11 - DNA, 51 bp, Artificial Sequence
  Synthetic Primer
ggcattcctttctcgggatcgtgcgtcatgaccaccacactgttgcccggc

SEQ ID NO: 12 - DNA, 32 bp, Artificial Sequence
  Synthetic Primer
ccctctagatcagtggtcgctgggcgcgcgat

SEQ ID NO: 13 - DNA, 34 bp, Artificial Sequence
  Synthetic Primer
cccggatccatgaccaccacactgttgcccggca

SEQ ID NO: 14 - DNA, 31 bp, Artificial Sequence
  Synthetic Primer
gcgctcgaggtggtcgctgggcgcgcgatgg

SEQ ID NO: 15 - DNA, 28 bp, Artificial Sequence
  Synthetic Primer
cccggatcccgtcccactacgacggccc

SEQ ID NO: 16 - DNA, 43 bp, Artificial Sequence
  Synthetic Primer
tggtggtgcgacgagcccatggcattcctttctcgggatcgtg

SEQ ID NO: 17 - DNA, 43 bp, Artificial Sequence
  Synthetic Primer
cacgatcccgagaaaggaatgccatgggctcgtcgcaccacca

SEQ ID NO: 18 - DNA, 29 bp, Artificial Sequence
  Synthetic Primer
ccctctagatcagtgcttggcctggccct

SEQ ID NO: 19 - DNA, 53 bp, Artificial Sequence
  Synthetic Primer
ccctctagatcagtggtggtggtggtggtggacatgtgccacctccggaacct

SEQ ID NO: 20 - DNA, 42 bp, Artificial Sequence
  Synthetic Primer
gttcacagtggcgcgaccgtgcttcctgcaccacgggcgagg

SEQ ID NO: 21 - DNA, 42 bp, Artificial Sequence
  Synthetic Primer
cctcgcccgtggtgcaggaagcacggtcgcgccactgtgaac

SEQ ID NO: 22 - DNA, 46 bp, Artificial Sequence
  Synthetic Primer
ggggcatttgctccgaaatgtttcggcttgaaccgacttcctgcac

SEQ ID NO: 23 - DNA, 46 bp, Artificial Sequence
  Synthetic Primer
gtgcaggaagtcggttcaagccgaaacatttcggagcaaatgcccc

SEQ ID NO: 24 - DNA, 44 bp, Artificial Sequence
  Synthetic Primer
gtccgtggccagttcctgactctgttcacagtggcgcgaccgtg

SEQ ID NO: 25 - DNA, 44 bp, Artificial Sequence
  Synthetic Primer
cacggtcgcgccactgtgaacagagtcaggaactggccacggac

SEQ ID NO: 26 - DNA, 59 bp, Artificial Sequence
  Synthetic Primer
tcgcccgtggtgcaggaagtcgggccaagccgacacggtcgcgccactgtgaacatttc

SEQ ID NO: 27 - DNA, 59 bp, Artificial Sequence
  Synthetic Primer
gaaatgttcacagtggcgcgaccgtgtcggcttggcccgacttcctgcaccacgggcga

SEQ ID NO: 28 - DNA, 59 bp, Artificial Sequence
  Synthetic Primer
tcgcccgtggtgcaggaagtcggtttgagccgacacggtcgcgccactgtgaacatttc

SEQ ID NO: 29 - DNA, 59 bp, Artificial Sequence
  Synthetic Primer
gaaatgttcacagtggcgcgaccgtgtcggctcaaaccgacttcctgcaccacgggcga

SEQ ID NO: 30 - DNA, 59 bp, Artificial Sequence
  Synthetic Primer
tcgcccgtggtgcaggaagtcggttcactccgacacggtcgcgccactgtgaacatttc

SEQ ID NO: 31 - DNA, 59 bp, Artificial Sequence
  Synthetic Primer
gaaatgttcacagtggcgcgaccgtgtcggagtgaaccgacttcctgcaccacgggcga

SEQ ID NO: 32 - DNA, 59 bp, Artificial Sequence
  Synthetic Primer
tcgcccgtggtgcaggaagtcggttcaagtagacacggtcgcgccactgtgaacatttc

SEQ ID NO: 33 - DNA, 59 bp, Artificial Sequence
  Synthetic Primer
gaaatgttcacagtggcgcgaccgtgtctacttgaaccgacttcctgcaccacgggcga

SEQ ID NO: 34 - DNA, 59 bp, Artificial Sequence
  Synthetic Primer
tcgcccgtggtgcaggaagtcggttcaagcctgcacggtcgcgccactgtgaacatttc

SEQ ID NO: 35 - DNA, 59 bp, Artificial Sequence
  Synthetic Primer
gaaatgttcacagtggcgcgaccgtgcaggcttgaaccgacttcctgcaccacgggcga

SEQ ID NO: 36 - DNA, 59 bp, Artificial Sequence
  Synthetic Primer
tcgcccgtggtgcaggaagtcggttcaagatagcacggtcgcgccactgtgaacatttc

SEQ ID NO: 37 - DNA, 59 bp, Artificial Sequence
  Synthetic Primer
gaaatgttcacagtggcgcgaccgtgctatcttgaaccgacttcctgcaccacgggcga

SEQ ID NO: 38 - DNA, 55 bp, Artificial Sequence
  Synthetic Primer
tcgcccgtggtgcaggaagtcggttcaggccgacacggtcgcgccactgtgaaca

SEQ ID NO: 39 - DNA, 55 bp, Artificial Sequence
  Synthetic Primer
tgttcacagtggcgcgaccgtgtcggcctgaaccgacttcctgcaccacgggcga

SEQ ID NO: 40 - DNA, 55 bp, Artificial Sequence
  Synthetic Primer
gcccgtggtgcaggaagtcggttcaagacgacacggtcgcgccactgtgaacatt

SEQ ID NO: 41 - DNA, 55 bp, Artificial Sequence
  Synthetic Primer
aatgttcacagtggcgcgaccgtgtcgtcttgaaccgacttcctgcaccacgggc

SEQ ID NO: 42 - DNA, 55 bp, Artificial Sequence
  Synthetic Primer
gcccgtggtgcaggaagtcggttcaagcagacacggtcgcgccactgtgaacatt

SEQ ID NO: 43 - DNA, 55 bp, Artificial Sequence
  Synthetic Primer
aatgttcacagtggcgcgaccgtgtctgcttgaaccgacttcctgcaccacgggc

SEQ ID NO: 44 - DNA, 55 bp, Artificial Sequence
  Synthetic Primer
tcgcccgtggtgcaggaagtcggttcaatccgacacggtcgcgccactgtgaaca

SEQ ID NO: 45 - DNA, 55 bp, Artificial Sequence
  Synthetic Primer
tgttcacagtggcgcgaccgtgtcggattgaaccgacttcctgcaccacgggcga

SEQ ID NO: 46 - DNA, 57 bp, Artificial Sequence
  Synthetic Primer
cccgtggtgcaggaagtcggttcaagcctacacggtcgcgccactgtgaacatttcg

SEQ ID NO: 47 - DNA, 57 bp, Artificial Sequence
  Synthetic Primer
cgaaatgttcacagtggcgcgaccgtgtaggcttgaaccgacttcctgcaccacggg

SEQ ID NO: 48 - DNA, 57 bp, Artificial Sequence
  Synthetic Primer
cccgtggtgcaggaagtcggttcaagccggcacggtcgcgccactgtgaacatttcg

SEQ ID NO: 49 - DNA, 57 bp, Artificial Sequence
  Synthetic Primer
cgaaatgttcacagtggcgcgaccgtgccggcttgaaccgacttcctgcaccacggg

SEQ ID NO: 50 - DNA, 47 bp, Artificial Sequence
  Synthetic Primer
gttcctgactctcatcgcggggcgctccgaaatgttcacagtggcgc

SEQ ID NO: 51 - DNA, 47 bp, Artificial Sequence
  Synthetic Primer
gcgccactgtgaacatttcggagcgccccgcgatgagagtcaggaac

SEQ ID NO: 52 - DNA, 51 bp, Artificial Sequence
  Synthetic Primer
cgcgccactgtgaacatttcggagcggatgccccgcgatgagagtcaggaa

SEQ ID NO: 53 - DNA, 51 bp, Artificial Sequence
  Synthetic Primer
ttcctgactctcatcgcggggcatccgctccgaaatgttcacagtggcgcg

SEQ ID NO: 54 - DNA, 48 bp, Artificial Sequence
  Synthetic Primer
acacggtcgcgccactgtgaacatcccggagcaaatgccccgcgatga

SEQ ID NO: 55 - DNA, 47 bp, Artificial Sequence
  Synthetic Primer
catcgcggggcatttgctccgggatgttcacagtggcgcgaccgtgt

SEQ ID NO: 56 - DNA, 54 bp, Artificial Sequence
  Synthetic Primer
tcgcgccactgtgaacatttcggagcaagtgccccgcgatgagagtcaggaact

SEQ ID NO: 57 - DNA, 54 bp, Artificial Sequence
  Synthetic Primer
agttcctgactctcatcgcggggcacttgctccgaaatgttcacagtggcgcga

SEQ ID NO: 58 - DNA, 54 bp, Artificial Sequence
  Synthetic Primer
tcgcgccactgtgaacatttcggagcaaacgccccgcgatgagagtcaggaact

SEQ ID NO: 59 - DNA, 54 bp, Artificial Sequence
  Synthetic Primer
agttcctgactctcatcgcggggcgtttgctccgaaatgttcacagtggcgcga

SEQ ID NO: 60 - DNA, 58 bp, Artificial Sequence
  Synthetic Primer
gaagtcggttcaagccgacacggttatatcactgtgaacatttcggagcaaatgcccc

SEQ ID NO: 61 - DNA, 58 bp, Artificial Sequence
  Synthetic Primer
ggggcatttgctccgaaatgttcacagtgatataaccgtgtcggcttgaaccgacttc

SEQ ID NO: 62 - DNA, 56 bp, Artificial Sequence
  Synthetic Primer
aactggccacggacgagcctttcaaatataggacgcacgatcccgagaaaggaatg

SEQ ID NO: 63 - DNA, 56 bp, Artificial Sequence
  Synthetic Primer
cattcctttctcgggatcgtgcgtcctatatttgaaaggctcgtccgtggccagtt

SEQ ID NO: 64 - DNA, 58 bp, Artificial Sequence
  Synthetic Primer
gaagtcggttcaagccgacacggtggggccactgtgaacatttcggagcaaatgcccc

SEQ ID NO: 65 - DNA, 58 bp, Artificial Sequence
  Synthetic Primer
ggggcatttgctccgaaatgttcacagtggccccaccgtgtcggcttgaaccgacttc

SEQ ID NO: 66 - DNA, 56 bp, Artificial Sequence
  Synthetic Primer
aactggccacggacgagcctttcaagccccggacgcacgatcccgagaaaggaatg

SEQ ID NO: 67 - DNA, 56 bp, Artificial Sequence
  Synthetic Primer
cattcctttctcgggatcgtgcgtccggggcttgaaaggctcgtccgtggccagtt

SEQ ID NO: 68 - DNA, 58 bp, Artificial Sequence
  Synthetic Primer
gaagtcggttcaagccgacacggtggagccactgtgaacatttcggagcaaatgcccc

SEQ ID NO: 69 - DNA, 58 bp, Artificial Sequence
  Synthetic Primer
ggggcatttgctccgaaatgttcacagtggctccaccgtgtcggcttgaaccgacttc

SEQ ID NO: 70 - DNA, 56 bp, Artificial Sequence
  Synthetic Primer
aactggccacggacgagcctttcaagctccggacgcacgatcccgagaaaggaatg

SEQ ID NO: 71 - DNA, 56 bp, Artificial Sequence
  Synthetic Primer
cattcctttctcgggatcgtgcgtccggagcttgaaaggctcgtccgtggccagtt

SEQ ID NO: 72 - DNA, 50 bp, Artificial Sequence
  Synthetic Primer
aagtcggttcaagccgacacggtcactgtgaacatttcggagcaaatgcc

SEQ ID NO: 73 - DNA, 50 bp, Artificial Sequence
  Synthetic Primer
ggcatttgctccgaaatgttcacagtgaccgtgtcggcttgaaccgactt

SEQ ID NO: 74 - DNA, 51 bp, Artificial Sequence
  Synthetic Primer
aactggccacggacgagcctttcaaggacgcacgatcccgagaaaggaatg

SEQ ID NO: 75 - DNA, 51 bp, Artificial Sequence
  Synthetic Primer
cattcctttctcgggatcgtgcgtccttgaaaggctcgtccgtggccagtt

SEQ ID NO: 76 - DNA, 55 bp, Artificial Sequence
  Synthetic Primer
aagtcggttcaagccgacacggtgctcgcactgtgaacatttcggagcaaatgcc

SEQ ID NO: 77 - DNA, 55 bp, Artificial Sequence
  Synthetic Primer
ggcatttgctccgaaatgttcacagtgcgagcaccgtgtcggcttgaaccgactt

SEQ ID NO: 78 - DNA, 56 bp, Artificial Sequence
  Synthetic Primer
aactggccacggacgagcctttcaacgagcggacgcacgatcccgagaaaggaatg

SEQ ID NO: 79 - DNA, 56 bp, Artificial Sequence
  Synthetic Primer
cattcctttctcgggatcgtgcgtccgctcgttgaaaggctcgtccgtggccagtt

SEQ ID NO: 80 - DNA, 52 bp, Artificial Sequence
  Synthetic Primer
gagtcaggaactggccacggacgagcctctcaagcgcgggacgcacgatccc

SEQ ID NO: 81 - DNA, 52 bp, Artificial Sequence
  Synthetic Primer
gggatcgtgcgtcccgcgcttgagaggctcgtccgtggccagttcctgactc

SEQ ID NO: 82 - DNA, 53 bp, Artificial Sequence
  Synthetic Primer
gagtcaggaactggccacggacgagttccttcaagcgcgggacgcacgatccc

SEQ ID NO: 83 - DNA, 53 bp, Artificial Sequence
  Synthetic Primer
gggatcgtgcgtcccgcgcttgaaggaactcgtccgtggccagttcctgactc

SEQ ID NO: 84 - DNA, 55 bp, Artificial Sequence
  Synthetic Primer
aagcgcgggacgcacgatcccgagagaggaatgccgtgcatatcgcagaaggcgt

SEQ ID NO: 85 - DNA, 56 bp, Artificial Sequence
  Synthetic Primer
acgccttctgcgatatgcacggcattcctctctcgggatcgtgcgtcccgcgcttg

SEQ ID NO: 86 - DNA, 55 bp, Artificial Sequence
  Synthetic Primer
caagcgcgggacgcacgatcccgaaggaaaatgccgtgcatatcgcagaaggcgt

SEQ ID NO: 87 - DNA, 55 bp, Artificial Sequence
  Synthetic Primer
acgccttctgcgatatgcacggcattttccttcgggatcgtgcgtcccgcgcttg

SEQ ID NO: 88 - DNA, 56 bp, Artificial Sequence
  Synthetic Primer
caagcgcgggacgcacgatcccgagagaggaatgccatgggctcgtcgcaccacca

SEQ ID NO: 89 - DNA, 56 bp, Artificial Sequence
  Synthetic Primer
tggtggtgcgacgagcccatggcattcctctctcgggatcgtgcgtcccgcgcttg

SEQ ID NO: 90 - DNA, 55 bp, Artificial Sequence
  Synthetic Primer
caagcgcgggacgcacgatcccgaaggaaaatgccatgggctcgtcgcaccacca

SEQ ID NO: 91 - DNA, 55 bp, Artificial Sequence
  Synthetic Primer
tggtggtgcgacgagcccatggcattttccttcgggatcgtgcgtcccgcgcttg

SEQ ID NO: 92 - DNA, 55 bp, Artificial Sequence
  Synthetic Primer
aagcgcgggacgcacgatcccgacttaggaatgccatgggctcgtcgcaccacca

SEQ ID NO: 93 - DNA, 55 bp, Artificial Sequence
  Synthetic Primer
tggtggtgcgacgagcccatggcattcctaagtcgggatcgtgcgtcccgcgctt

SEQ ID NO: 94 - DNA, 55 bp, Artificial Sequence
  Synthetic Primer
aagcgcgggacgcacgatcccgacttaggaatgccgtgcatatcgcagaaggcgt

SEQ ID NO: 95 - DNA, 55 bp, Artificial Sequence
  Synthetic Primer
acgccttctgcgatatgcacggcattcctaagtcgggatcgtgcgtcccgcgctt

SEQ ID NO: 96 - DNA, 49 bp, Artificial Sequence
  Synthetic Primer
aggaactggccacggacgagcctaagaagcgcgggacgcacgatcccga

SEQ ID NO: 97 - DNA, 49 bp, Artificial Sequence
  Synthetic Primer
tcgggatcgtgcgtcccgcgcttcttaggctcgtccgtggccagttcct

SEQ ID NO: 98 - DNA, 51 bp, Artificial Sequence
  Synthetic Primer
gacgcacgatcccgagaaaggaatgccatgagcgccctgctggccgcccac

SEQ ID NO: 99 - DNA, 51 bp, Artificial Sequence
  Synthetic Primer
gtgggcggccagcagggcgctcatggcattcctttctcgggatcgtgcgtc

SEQ ID NO: 100 - DNA, 53 bp, Artificial Sequence
  Synthetic Primer
gacgcacgatcccgagaaaggaatgccatgtcccgcctcgacaagtccaaggt

SEQ ID NO: 101 - DNA, 53 bp, Artificial Sequence
  Synthetic Primer
accttggacttgtcgaggcgggacatggcattcctttctcgggatcgtgcgtc

SEQ ID NO: 102 - DNA, 36 bp, Artificial Sequence
  Synthetic Primer
ggcaagcttttaggagccggactcgcacttgagctg

SEQ ID NO: 103 - DNA, 59 bp, Artificial Sequence
  Synthetic Primer
cccaagctttcagtggtggtggtggtggtgggcgttccgtgatccgggggccgtcggtc

SEQ ID NO: 104 - DNA, 54 bp, Artificial Sequence
  Synthetic Primer
cccaagctttcagtggtggtggtggtggtgtcgggccaccaccaggctgatcga

SEQ ID NO: 105 - DNA, 57 bp, Artificial Sequence
  Synthetic Primer
cccaagctttcagtggtggtggtggtggtgtcgggtttcctcatcggtgttggtggt

SEQ ID NO: 106 - DNA, 54 bp, Artificial Sequence
  Synthetic Primer
cccaagctttcagtggtggtggtggtggtggtggtcgctgggcgcgcgatggtc

SEQ ID NO: 107 - DNA, 51 bp, Artificial Sequence
  Synthetic Primer
gacgcacgatcccgagagaggaatgccatgagcgccctgctggccgcccac

SEQ ID NO: 108 - DNA, 51 bp, Artificial Sequence
  Synthetic Primer
gtgggcggccagcagggcgctcatggcattcctctctcgggatcgtgcgtc

SEQ ID NO: 109 - DNA, 53 bp, Artificial Sequence
  Synthetic Primer
gacgcacgatcccgagagaggaatgccatgtcccgcctcgacaagtccaaggt

SEQ ID NO: 110 - DNA, 53 bp, Artificial Sequence
  Synthetic Primer
accttggacttgtcgaggcgggacatggcattcctctctcgggatcgtgcgtc

SEQ ID NO: 111 - DNA, 19 bp, Artificial Sequence
  Synthetic Primer
caatgtcgtgttggagaag

SEQ ID NO: 112 - DNA, 18 bp, Artificial Sequence
  Synthetic Primer
cgccgatcttgtggtagg

SEQ ID NO: 113 - DNA, 20 bp, Artificial Sequence
  Synthetic Primer
ctcatcgtgctgatcttcca

SEQ ID NO: 114 - DNA, 22 bp, Artificial Sequence
  Synthetic Primer
gagcttcttgttgagcacatag

SEQ ID NO: 115 - DNA, 16 bp, Artificial Sequence
  Synthetic Primer
ggttccagccgctgtt

SEQ ID NO: 116 - DNA, 21 bp, Artificial Sequence
  Synthetic Primer
cccaggcagtagaagatgatg

SEQ ID NO: 117 - DNA, 18 bp, Artificial Sequence
  Synthetic Primer
ccatcgtggcttcgagac

SEQ ID NO: 118 - DNA, 17 bp, Artificial Sequence
  Synthetic Primer
ccaccaggctgatcgac

SEQ ID NO: 119 - DNA, 17 bp, Artificial Sequence
  Synthetic Primer
tgcaccagatgcgtgac

SEQ ID NO: 120 - DNA, 19 bp, Artificial Sequence
  Synthetic Primer
tcacagacgatagcgacct

SEQ ID NO: 121 - DNA, 20 bp, Artificial Sequence
  Synthetic Primer
ccaggaggagatcaaccaac

SEQ ID NO: 122 - DNA, 17 bp, Artificial Sequence
  Synthetic Primer
gcccgaagacgaacgag

SEQ ID NO: 123 - DNA, 32 bp, Artificial Sequence
  Synthetic Primer
ggcaagctttcagtggtcgctgggcgcgcgat

SEQ ID NO: 124 - DNA, 35 bp, Artificial Sequence
  Synthetic Primer
gcgcctgcaggcgtcccactacgacggccccacgg

SEQ ID NO: 125 - DNA, 198 bp, Artificial Sequence
  Synthetic Polynucleotide
tgggggcgatttctgccgggaatgcgtccccgttatactgctgtggacaacctcgcccgtggtgcaggaagtcggttcaagccgacacggtcgcgccactgtgaacatttcggagcaaatgccccgcgatgagagtcaggaactggccacggacgagcctttcaagcgcgggacgcacgatcccgagaaaggaatgcc

SEQ ID NO: 126 - DNA, 198 bp, Artificial Sequence
  Synthetic Polynucleotide
ggcattcctttctcgggatcgtgcgtcccgcgcttgaaaggctcgtccgtggccagttcctgactctcatcgcggggcatttgctccgaaatgttcacagtggcgcgaccgtgtcggcttgaaccgacttcctgcaccacgggcgaggttgtccacagcagtataacggggacgcattcccggcagaaatcgccccca

SEQ ID NO: 127 - DNA, 200 bp, Artificial Sequence
  Synthetic Polynucleotide
cuguagcauccacuugccgguccugugaguuaauagggaauccagugcgaaucuggagcugacgcgcagcgguaaggaaaggugcgaugauugcguuaugcggacacugccauccggugggaagucaucaucucuuaguaucuuagauaccccuccaagcccgaagaccugccggccaacgucucaucaucgcguaauau

SEQ ID NO: 128 - DNA, 214 bp, Artificial Sequence
  Synthetic Polynucleotide
cgguagcauccgugggccggccugugaguuaauagggaauccagugaaaaucuggagcugacgcgcagcgguaaggaaaggugagaugagagcguaagcagacacugccuccggcgggaagucaucauuucugcuauccagccaacggauaacccuccaagcccgaagaccugccgcuaacgucgcaucugguuuuucaucaucgcguacuauc

SEQ ID NO: 129 - DNA, 153 bp, Artificial Sequence
  Synthetic Polynucleotide
gaaaaucccccgcagccccccugcugugaugcugacgaccccguaaagaccacugaucgcaagauugggaaggacgggcgaggaggacgcuaagccagaagaccugccugucggugauaaccaacaacuucggugggaaguggguugcgaaga

SEQ ID NO: 130 - DNA, 142 bp, Artificial Seuqence
  Synthetic Polynucelotide
ucaagccgacacggucgcgccacugugaacauuucggagcaaaugccccgcgaugagagucaggaacuggccacggacgagccuuucaagcgcgggacgcacgaucccgagaaaggaaugccggaaugccgugcauaucgca

SEQ ID NO: 131 - DNA, 157 bp, Artificial Sequence
  Synthetic Polynucleotide
gcgaauccggaacugucccgcaacgguguacuugcgugcauccguacgucuucgcacgugcgcgcacgccuguccaguccgaggaccugccgacagugcgcccggccgcccuggcccgggugccaugacguccgggccucgcggagugggccggugg

(19) United States
(12) Patent Application Publication
(10) Pub. No.: US 2022/0364135 A1
(43) Pub. Date: Nov. 17, 2022

(54) MODIFIED PROPIONIBACTERIUM AND METHODS OF USE

(71) Applicant: UNIVERSITY OF FLORIDA RESEARCH FOUNDATION, INCORPORATED, Gainesville, FL (US)

(72) Inventor: Mansour MOHAMADZADEH, San Antonio, TX (US)

(21) Appl. No.: 17/637,583

(22) PCT Filed: Aug. 28, 2020
(86) PCT No.: PCT/US20/48324

§ 371 Date: Feb. 23, 2022

Related U.S. Application Data
(60) Provisional application No. 62/894,301, filed on Aug. 30, 2019.

(51) Int. Cl. C12P 19/42; C07K 14/195; C12N 1/20

(52) U.S. Cl. CPC C12P 19/42; C07K 14/195; C12N 1/20

(57) ABSTRACT

The present disclosure is directed to modified Propionibacterium and methods of use (e.g., to produce vitamin B12). Methods of increasing vitamin B12 production in a bacterium comprising a vitamin B12 riboswitch are also contemplated.

### CROSS-REFERENCE TO RELATED APPLICATIONS

The present application claims the benefit of priority to U.S. Provisional Application No. 62/894,301, filed Aug. 30, 2019, the disclosure of which is incorporated herein by reference in its entirety.

### STATEMENT OF GOVERNMENT SUPPORT

This invention was made with government support under DK109560 awarded by The National Institutes of Health. The government has certain rights in the invention.

### FIELD OF THE INVENTION

The present disclosure is directed to modified Propionibacterium and methods of use, including use to produce vitamin B12. Methods of increasing vitamin B12 production in a bacterium comprising a vitamin B12 riboswitch are also contemplated.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named 54547_Seqlisting.txt; Size: 26,555 bytes, created on Aug. 27, 2020.

### BACKGROUND

Propionibacteria are gram-positive, facultative anaerobic, non-motile microorganisms with high GC contents, which can be taxonomically classified into the cutaneous (e.g., Propionibacterium acnes) and dairy bacterial species (e.g., Propionibacterium freudenreichh) (20, 21). P. freudenreichii, along with Pseudomonas species, is the major producer of vitamin B12 (VB12) and thus, has been widely used for industrial fermentation (2, 17).

Vitamin B12 (VB12), also known as cobalamin, is uniquely synthesized by some bacteria and archaebacteria, and is a crucial cofactor for critical enzymes catalyzing numerous transmethylation and the biochemical reactions(1, 2). VB12 structurally consists of a corrin ring, in which the cobalt is positioned centrally and coordinated with upper and lower ligand made-up of 5,6-dimethylbenzimidazole (DMB) (1, 3, 4). Both in bacterial and mammalian cells, methionine synthase for biosynthesis of S-adenosylmethionine (SAM), and methylmalonyl-CoA mutase converting methylmalonyl-CoA to succinyl-CoA, are highly dependent on VB12 for their metabolic activities (5). This renders VB12 to be essentially required for the biosynthesis of nucleic acids and the amino and fatty acid metabolisms. Henceforth, VB12 deficiency is critically associated with micronuclei formation and chromosomal abnormalities (6-8) and highly contributes to adverse pregnancy along with the neurological morbidity and death of neonates (9-14).

### SUMMARY

In one aspect, described herein is a Propionibacterium that has been modified to overproduce vitamin B12. In some embodiments, the Propionibacterium is P. freudenreichii. In some embodiments, the Propionibacterium is P. UF1. In some embodiments, the Propionibacterium comprises a mutation in a vitamin B12 riboswitch of the bacterium. The mutation decreases the activity of the vitamin B12 riboswitch. In some embodiments, the vitamin B12 riboswitch is cbiMCbl. In some embodiments, the mutation results in a deletion of stem loop 1 (SL1) of the cbiMCbl riboswitch. In some embodiments, the vitamin B12 riboswitch retains sequences encoding stem loops 2 and 3 of the vitamin B12 riboswitch.

Compositions comprising a modified Propionibacterium that has been modified to overproduce vitamin B12 are also contemplated. In some embodiments, the composition is a food product. In some embodiments, the composition is a beverage.

In another aspect, described herein is a method of increasing vitamin B12 production in a bacterium, the method comprising: (a) providing a bacterium comprising a vitamin B12 riboswitch; and (b) introducing a mutation in a vitamin B12 riboswitch that decreases activity of the vitamin B12 riboswitch, thereby increasing vitamin B12 production. In some embodiments, the method further comprises culturing the bacterium under conditions sufficient to produce vitamin B12. In some embodiments, the method further comprises isolating the vitamin B12 produced from the bacterium. In some embodiments, the bacterium is Pseudomonas dentrificans, Rhodobacter capusulatus, Rhodobacter sphaeroides, Sinorhizobium meliloti, Salmonella typhimurium, Bacillus megaterium, Propionibacterium shermanii, Thermotoga sp. RQ2, Thermotoga maritima MSB8, Thermotoga neapolitana, Thermotoga petrophila, Escherichia coli, Thermotoga naphthophila, Thermotoga thermarum, Thermotoga lettingae, Fervidobacterium nodosum, Thermosipho melanesiensis, Thermosipho africanus, Kosmotoga olearia, Mesotoga prima, Petrotoga mobilis. In some embodiments, the bacterium is Propionibacterium shermanii or Sinorhizobium meliloti. In some embodiments, the bacterium is Propionibacterium. In some embodiments, the bacterium is P. freudenreichii. In some embodiments, the bacterium is P. UF1. In some embodiments, the vitamin B12 riboswitch is cbiMCbl. In some embodiments, the mutation is deletion of stem loop 1 (SL1) of the cbiMCbl riboswitch. In some embodiments, the bacterium does not naturally produce vitamin B12 but has been genetically engineered to produce vitamin B12 (e.g., Escherichia coli and Pseudomonas dentrificans).

In another aspect, described herein is a method of producing vitamin B12, the method comprising (a) culturing a modified Propionibacterium described herein under conditions sufficient to produce vitamin B12; and (b) isolating vitamin B12 from the bacterium. In some embodiments, the bacterium is P. freudenreichii. In some embodiments, the bacterium is P. UF1.

In another aspect, described herein is a method of increasing vitamin B12 production in the gut of a mammalian subject comprising administering to the subject a composition described herein.

In yet another aspect, described herein is a method of increasing vitamin B12 production in the gut of a mammalian subject comprising administering to the subject a composition comprising a Propionibacterium and an agent that inhibits the activity of a vitamin B12 riboswitch in the Propionibacterium. In some embodiments, the vitamin B12 riboswitch is cbiMCbl.

In the following detailed description, embodiments are described in sufficient detail to enable those skilled in the art to practice them, and it is to be understood that other embodiments may be utilized and that changes may be made without departing from the spirit and scope of the present subject matter. The following detailed description is, therefore, not to be taken in a limiting sense, and the scope of embodiments is defined only by the appended claims. Also herein, the recitations of numerical ranges by endpoints include all numbers subsumed within that range (e.g., 1 to 5 includes 1, 1.5, 2, 2.75, 3, 3.80, 4, 5, and the like). Furthermore, disclosure of a range includes disclosure of all subranges included within the broader range (e.g., 1 to 5 discloses 1 to 4, 1.5 to 4.5, 4 to 5, and the like).

### BRIEF DESCRIPTION OF THE FIGURES

FIGS. 1A-1E show that cobA is essential for vitamin B12 (VB12) biosynthesis in P.UF1. FIG. 1A provides the proposed biosynthetic pathway for VB12 in P. UF1. cobA is responsible for converting uroporphyrinogen III to precorrin-2. FIG. 1B provides the genetic organization of P. UF1, ΔcobA P. UF1 and C-ΔcobA P. UF1 strains. cmR, chloramphenicol resistant gene. hygB, hygromycin B. FIG. 1C is a gel showing the PCR identification of P. UF1, ΔcobA P. UF1 and C-ΔcobA P.UF1 using primers P1/P2 and P3/P4 as shown in B. FIG. 1D is a gel showing Western blot (WB) analysis of cobA expression in P. UF1, ΔcobA P. UF1 and C-ΔcobA P. UF1, using mouse serum antibodies against CobA. The large surface layer protein (LspA) was used as a reference control. FIG. 1E provides HPLC chromatograms of VB12 extracted from P. UF1, ΔcobA P. UF1, and C-ΔcobA P. UF1. Bar graph showing the intracellular levels of VB12 in the indicated strains. Data are representative of three independent experiments. Error bars indicate SEM. **p<0.01, ****p<0.00001, 2-tailed unpaired t test.

FIG. 2 is a graph demonstrating that cobA is an essential gene for VB12 biosynthesis by P.UF1. Time-course analysis of VB12 production by P. UF1 and ΔcobA P. UF1 in MRS and Poznan medium.

FIGS. 3A-3F demonstrates that CobA critically impacts the transcriptome and metabolome of P. UF1. FIG. 3A is a PCA plot derived from the transcriptomes of P. UF1 and ΔcobA P. UF1 harvested at day 3. FIG. 3B provides pathway analysis of stringently significant genes (p<0.05; fold change >2) of ΔcobA P. UF1 versus P. UF1. Red dashed line shows the permutation p value of 0.05. FIG. 3C is a heatmap showing a selection of top differentially expressed genes (p<0.05, fold change >1.5). FIG. 3D is a PCA plot derived from the metabolomes of P. UF1 and ΔcobA P. UF1 harvested at day 3 and 6. FIG. 3D provides metabolic pathway analysis of P. UF1 versus ΔcobA P. UF1 harvested at day 3. Red dashed line shows the permutation p value of 0.05 using Mummichog. FIG. 3E provides scatter plots for selected metabolite features with putative annotation. The m/z, retention time (RT, in seconds) and adduct ion are labeled for each metabolite. ** p<0.01, **** p<0.00001.

FIGS. 4A-4H show that the cobA operon is feedback-regulated by vitamin B12 (VB12). FIG. 4A is a graph showing that the intracellular levels of VB12 and FbFP fluorescence intensity in FbFP-WT strain cultured with VB12 or DMB plus cobalt at day 10.

FIG. 4B provides a graph and gel showing that the intracellular levels of VB12 and cbiM expression in CbiM-WT strain cultured with VB12 or DMB plus cobalt at day 10. FIG. 4C provides graphs showing the FbFP fluorescence intensity of FbFP-ΔcobA strain incubated with increasing VB12 (0-2500 ng/ml) (Left graph). Dose-response curve of VB12 on the FbFP expression by FbFP-ΔcobA strain (Right graph). FIG. 4D is a gel showing the Western Blot analysis of cbiM expression of CbiM-ΔcobA strain incubated with increasing VB12 (0-2500 ng/ml) using anti-HIS antibody. FIG. 4E is a gel showing the Western Blot analysis of cobA expression of P. UF1 incubated with increasing VB12 (0-25000 ng/ml) using mouse serum antibodies against CobA. FIG. 4F depicts the genetic organization of the cobA operon (top) and analysis of cbiN, cbiO and cobA expression responding to different concentrations of VB12 (0-25000 ng/ml) using anti-HIS antibody (bottom). FIG. 4G is a graph showing the FbFP fluorescence intensity of FbFP-ΔcobA strain incubated with different VB12 analogues. Acbl: adenosylcobalamin, Ccbl: Cyanocobalamin, Hcbl: Hydroxocobalamin, Mcbl: Methylcobalamin. FIG. 3H is a gel showing the Western Blot analysis of cbiM expression in CbiM-ΔcobA strain incubated with different VB12 analogues using anti-His antibody. Data are representative of three independent experiments (4A-4C and 4G). Error bars indicate SEM. **** p<0.0001, 2-tailed unpaired t test.

FIGS. 5A-5G demonstrate that stem-loops of cbiMCbl riboswitch are key for vitamin B12 (VB12)-mediated gene regulation. FIG. 5A depicts the secondary structure of cbiMCbl riboswitch predicted using the forna software. FIG. 5B is a graph showing the FbFP fluorescence intensity in FbFP-ΔcobA strain harboring WT or SL1-deleted riboswitch cultured with VB12 (0-5000 ng/ml). FIG. 5C is a graph showing the dose-response curve of VB12 on the FbFP expression. FIG. 5D provides gels showing the anti-HIS Western Blot analysis of cbiM expression in CbiM-ΔcobA strain with WT or SL1-deleted riboswitch in the presence of VB12 (0-5000 ng/ml). FIGS. 5E and 5F provide a graph and a gel, respectively, showing the FbFP fluorescence intensity and cbiM expression driven by SL1-mutated riboswitches in response to VB12 (0, 250, 2500 ng/ml). FIGS. 5G and 5H provide a graph and a gel, respectively, showing the FbFP fluorescence intensity and cbiM expression directed by SL3-mutated riboswitches with various concentrations of VB12. Data are representative of three independent experiments (FIGS. 5B, 5C, 5E and 5G). Error bars indicate SEM.

FIGS. 6A-6C show the key stem-loops of cbiMCbl riboswitch. FIG. 6A shows the FbFP fluorescence intensity of FbFP-ΔcobA strain incubated with various concentrations of VB12, wild-type, SL1, SL2 and SL3 deleted mutant cbiMCbl riboswitch. FIG. 6B shows the results of Western Blot analysis of cbiM expression of CbiM-ΔcobA strain incubated with various concentrations of VB12 using anti-His antibody, wild-type, SL1, SL2 and SL3 deleted mutant cbiMCbl riboswitch. FIG. 6C shows the architecture of expression units and cbiMCbl riboswitch in 5′ UTR of cobA operon, transcription start site (TSS). Data are representative of three independent experiments (FIG. 6A). Error bars indicate mean SEM.

FIGS. 7A-7F demonstrate that cbiMCbl riboswitch regulates the translation of cobA operon. FIG. 7A provides a multiple alignment of VB12 riboswitches. Shaded background denotes P0 and P3 stems and their complementary sequences (P0′ and P3′). Conserved core region is shown in italic, bold and black letters. RBSs are shown in bold and black letters. The underlining denotes the proposed antisequester and RBS-sequester. Purple underlines denote the candidate pseudoknots (Pkn) and complementary pseudoknots (Pkn′). EC: Escherichia coli, ST: Salmonella Typhimurium, SC: Streptomyces coelicolor. The summary table showing the sequences of respective mutations. FIGS. 7B and 7C provides a graph and a gel, respectively, showing the FbFP fluorescence intensity and WB analysis of cbiM expression by a series of riboswitches mutated in Pkn, Pkn′, sequester, or antisequester. FIGS. 7D and 7E provide a graph and a gel, respectively showing Western Blot analysis of cbiM expression and FbFP fluorescence intensity by riboswitches with paired double mutations in Pkn:Pkn′ or sequester:antisequester. FIG. 7F provides a gel showing the Western Blot analysis of cbiO and tetR expression by WT and the double mutant riboswitch R:AR3. Data are representative of three independent experiments (FIGS. 7B and 7E). Error bars indicate SEM.

FIG. 8 shows that cbiMCbl riboswitch controls cobA operon transcription and VB12 production. FIG. 8A is a graph showing the results of qRT-PCR analysis of expression of cbiMNQO and cobA genes in P. UF1 incubated with various concentrations of VB12. FIG. 8B is a graph showing the results of HPLC analysis of VB12 production by WT and SL1-deleted riboswitches within OW-operon and OΔSL1-operon strains. The bacteria-absorbed VB12 was excluded for the analysis. Data are representative of three independent experiments. Error bars indicate SEM. * p<0.05, ** p<0.01, *** p<0.001, 2-tailed unpaired t test.

### DETAILED DESCRIPTION

Gut bacteria-associated metabolites maintain host immune and developmental homeostasis. As shown in the Examples herein, the cbiMCbl riboswitch regulates the transcriptional and translational machineries of the cobA operon, controlling bacterial vitamin B12 (VB12) biosynthesis. As demonstrated herein, molecular modification of this riboswitch significantly enhanced VB12 production within the commensal P. UF1.

In one aspect, described herein is a bacterium that has been modified to overproduce vitamin B12. The term “modified bacterium” refers to a bacterium altered, modified or engineered (e.g., genetically engineered) such that it exhibits an altered, modified or different genotype or phenotype (e.g., when the genetic modification affects coding nucleic acid sequences of the bacterium) as compared to the naturally-occurring or parent bacterium from which it was derived.

In some embodiments, the bacterium is P. freudenreichii. In some embodiments, the bacterium is modified to include a mutation in a vitamin B12 riboswitch, wherein the mutation decreases activity of the vitamin B12 riboswitch. As described in the Examples, introducing a mutation in the vitamin B12 riboswitch decreases activity of the vitamin B12 riboswitch, which results in overproduction of vitamin B12. Mutating the region of the bacterial genome that encodes the vitamin B12 riboswitch can include, for example, deletion, substitution or insertion of nucleic acids within the genome of the bacterium encoding the riboswitch to generate a mutant riboswitch with decreased activity.

In some embodiments, the mutation is deletion of a stem loop of the riboswitch. In some embodiments, the mutation is deletion of stem loop1 1 (SL1) of the riboswitch. In some embodiments the vitamin B12 riboswitch is cbiMCbl.

The term “overproduction” as used herein refers to the ability of a bacterium to produce a product (e.g., a metabolite such as vitamin B12) at a higher level than is normally produced in the bacterium, thereby resulting in a greater amount of product than normally found in the bacterium. In this regard, overproduction is achieved by, for instance and without limitation, deleting stem loop 1 (SL1) in the vitamin B12 riboswitch, thereby increasing vitamin B12 production. An exemplary method of introducing a mutation in a vitamin B12 riboswitch is described in the Examples.

It will be appreciated that other bacterium comprise vitamin B12 riboswitches, and that the methods described herein are applicable to other bacterium. For example, in some embodiments, the bacterium is In some embodiments, the bacterium is Pseudomonas dentrificans, Rhodobacter capusulatus, Rhodobacter sphaeroides, Sinorhizobium meliloti, Salmonella typhimurium, Bacillus megaterium, Propionibacterium shermanii, Thermotoga sp. RQ2, Thermotoga maritima MSB8, Thermotoga neapolitana, Thermotoga petrophila, Escherichia coli, Thermotoga naphthophila, Thermotoga thermarum, Thermotoga lettingae, Fervidobacterium nodosum, Thermosipho melanesiensis, Thermosipho africanus, Kosmotoga olearia, Mesotoga prima, Petrotoga mobilis. In some embodiments, the bacterium is Propionibacterium shermanii or Sinorhizobium meliloti. In some embodiments, the bacterium is Propionibacterium. In some embodiments, the bacterium is P. freudenreichii. In some embodiments, the bacterium is P. UF1. In some embodiments, the vitamin B12 riboswitch is cbiMCbl. In some embodiments, the mutation is deletion of stem loop 1 (SL1) of the cbiMCbl riboswitch. In some embodiments, the bacterium does not naturally produce vitamin B12 but has been genetically engineered to produce vitamin B12 (e.g., Escherichia coli and Pseudomonas dentrificans). See Fang et al., Microb. Cell Fact, 16:15, 2017; Fang et al., Nat. Commun. 9:4917, 2018 and Xia et al., Bioprocess. Biosyst. Eng. 38:1065-73, 2015, the disclosures of which are incorporated herein by reference in their entireties.

A riboswitch is a region in an mRNA molecule that can directly bind a small target molecule, the binding of which modulates production of the encoded gene product. The small target molecules include, among others, vitamins, amino acids and nucleotides, and the binding is selective through a conserved sensor domain. Upon binding, the conformation of a variable “expression platform” coupled to the sensor domain is changed, and this can affect different modes of gene expression control including transcription termination, translation initiation or mRNA processing. Notably, riboswitches exert their functions without the need for protein cofactors. In most cases, they act in feedback regulation mechanisms: once the level of an end product (e.g., vitamin B12) in a metabolic pathway rises, riboswitch binding occurs, triggering a repression of gene expression in the same pathway. The substrate specificity of riboswitches is extremely high, allowing them to perform their activity amid the presence of numerous related compounds.

### Vitamin B12 Production and Isolation

Another aspect of the disclosure is directed to a method of increasing vitamin B12 in a bacterium. This method comprises introducing a mutation in the vitamin B12 riboswitch that decreases activity of the vitamin B12 riboswitch and culturing the bacterium under conditions sufficient to produce vitamin B12.

A variety of culture systems are known in the art, including T-flasks, spinner and shaker flasks, roller bottles and stirred-tank bioreactors. Roller bottle cultivation is generally carried out by seeding cells into roller bottles that are partially filled (e.g., to 10-30% of capacity) with medium and slowly rotated, allowing cells to attach to the sides of the bottles and grow to confluency. The cell medium is harvested by decanting the supernatant, which is replaced with fresh medium. Anchorage-dependent cells can also be cultivated on microcarrier, e.g., polymeric spheres, that are maintained in suspension in stirred-tank bioreactors. Alternatively, cells can be grown in single-cell suspension.

Culture medium may be added in a batch process, e.g., where culture medium is added once to the cells in a single batch, or in a fed batch process in which small batches of culture medium are periodically added. Medium can be harvested at the end of culture or several times during culture. Continuously perfused production processes are also known in the art, and involve continuous feeding of fresh medium into the culture, while the same volume is continuously withdrawn from the reactor. Perfused cultures generally achieve higher cell densities than batch cultures and can be maintained for weeks or months with repeated harvests.

Host cells may be cultured using standard media well known to the skilled artisan. The media will usually contain all nutrients necessary for the growth and survival of the cells. Typically, an antibiotic or other compound useful for selective growth of transformed cells is added as a supplement to the media. The compound to be used will often be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline, geneticin, and neomycin.

The amount of vitamin B12 produced by a host cell can be evaluated using standard methods known in the art. Such methods include, without limitation, enzyme-linked immunosorbent assays (ELISAs), capillary electrophoresis (CE), radioisotope and mass spectrometry High Performance Liquid Chromatography (HPLC) separation, chemiluminescence (CL), absorption, fluorescence, surface plasmon resonance (SPR) and Raman spectroscopy.

Vitamin B12 produced by the bacterial cell can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification, Academic Press, Inc. N.Y. (1990)).

In another aspect, the increased production of vitamin B12 occurs in vivo (e.g., in the gut of a mammalian subject). In that regard, in one embodiment, the method comprises administering or ingesting a modified bacterium having a mutated vitamin B12 riboswitch, such as the modified bacteria described herein. Alternatively, the method comprises administering (or ingesting) an unmodified bacterium that produces vitamin B12 (e.g., a Propionibacterium such as P. UF1) in combination with an agent that inhibits the activity of a vitamin B12 riboswitch in the bacterium. Exemplary agents include small molecules and inhibitory RNA (RNAi) In some embodiments, the bacterium and the agent are administered (or ingested) at the same time. In some embodiments, the bacterium and the agent are administered sequentially (e.g., at separate times). In some embodiments, the bacterium and the agent are administered within minutes (e.g., about 1 minutes, about 5 minutes, about 10 minutes, about 20 minutes, about 30 minutes, about 40 minutes, about 50 minutes) or hours (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours, about 36 hours or about 48 hours) of each other.

### Compositions, Routes of Administration, Dose

Compositions comprising the modified bacterium described herein are also contemplated. In various aspects, the composition comprises live cultures of the bacteria, freeze-dried bacteria, or killed bacteria comprising the vitamin B12. The disclosure herein relating to compositions of modified bacteria also apply to compositions of unmodified bacteria, such as the unmodified bacteria described above.

In some embodiments, the composition can be in the form of capsules, pills, tablets, powders, granules, or as a solution or a suspension in an aqueous or non-aqueous liquid, or as an elixir or syrup, etc. In some embodiments, the composition is a food product, such as a probiotic food product. A “probiotic food product” refers to a food product which contains microorganisms associated with beneficial effects to humans and animals upon ingestion. Optionally, the food product is a beverage. The food or drink product may be a children's nutritional product such as a follow-on formula, beverage, milk, yogurt, fruit juice, fruit-based drink, chewable tablet, cookie, cracker, or a milk powder or the product may be an infant's nutritional product, such as an infant formula.

Compositions described herein can be administered by any means to deliver the composition to the intestine of a subject (e.g., oral administration, ingestion, or instillation directly into the intestines of the subject).

The modified bacterium (or composition comprising the modified bacterium) is administered to (or ingested by) a subject in need thereof in an effective amount which achieves a desired biological response in a clinically relevant time frame. In some embodiments, a bacterium that produces vitamin B12 (e.g., a Propionibacterium such as P. UF1) or composition comprising the bacterium is administered to (or ingested by) a subject in need thereof in combination with an agent that inhibits the activity of a vitamin B12 riboswitch in the bacterium.

An “effective amount” refers to the amount of the bacterium (whether modified or unmodified in combination with an agent that inhibits the activity of the vitamin B12 riboswitch in the bacterium), for example, P. freudenreichii, which is effective in producing beneficial effects in a subject ingesting the bacterium compared to a subject not exposed to the bacterium. In some embodiments, the beneficial effect is host intestinal homeostasis.

An effective amount of a modified bacterium described herein (or unmodified bacterium in combination with an agent that inhibits the activity of the vitamin B12 riboswitch in the bacterium) is determined based on the intended goal and may vary with the subject to which the composition is administered. The term “unit dose” refers to a physically discrete unit suitable for use in a subject, each unit containing a predetermined quantity of the composition calculated to produce the beneficial effect in association with its administration, for example, the appropriate route and treatment regimen. Generally, the dosage of the modified bacterium (or unmodified bacterium in combination with an agent that inhibits the activity of the vitamin B12 riboswitch in the bacterium) will vary depending upon such factors as the subject's age, weight, height, sex, general medical condition and medical history.

The effective amount of a bacterium can be expressed as an absolute number, for example, colony forming units (CFU), or as a body weight based dosage, for example CFU/Kg of body weight of the subject. Typically, an effective amount of the bacterium is about 10⁴ to about 10¹² CFU, about 10⁵ to about 10¹¹ CFU, about 10⁶ to about 10¹⁰ CFU, about 10⁸ to about 10¹⁰ CFU or about 10⁸ to about 10¹² CFU. In a specific embodiment, the effective amount is about 10⁴ to about 10¹² CFU/Kg, about 10⁵ to about 10¹¹ CFU/Kg, about 10⁶ to about 10¹⁰ CFU/Kg, about 10⁸ to about 10¹⁰ CFU/Kg or about 10⁸ to about 10¹² CFU/Kg of the body weight of the subject to which the composition is administered. In one embodiment, the effective amount is about 10¹² to about 10¹³ CFU/Kg of the body weight of the subject to which the composition is administered.

In some embodiments, an effective amount comprises administration of multiple doses of a bacterium. The pharmaceutically effective amount may comprise 1, 2, 3, 4, 5, 6, 7, 8, 9, 10, 15, 20, 25, 30, 35, 40, or more doses of a composition comprising a bacterium. In some embodiments, doses are administered over the course of 1 day, 2 days, 3 days, 4 days, 5 days, 6 days, 7 days, 10 days, 14 days, 21 days, 30 days, or more than 30 days. Moreover, treatment of a subject with a therapeutically effective amount of a bacterium can include a single treatment or can include a series of treatments. It will also be appreciated that the effective dosage of a bacterium used for treatment may increase or decrease over the course of a particular regimen.

### EXAMPLES

Materials and Methods:

Bacterial strains and growth: Escherichia coli NEB 5-alpha (New England BioLabs, MA) used for plasmid construction and E. coli Rosetta (DE3) (Sigma-Aldrich, St. Louis, Mo.) used for protein expression were grown in Luria-Bertani (LB) medium at 37° C. The P. UF1 and its genetically modified strains were grown at 30° C. in MRS medium (Difco Laboratories, Detroit, Mich.) with 1% (w/v) sodium lactate (Thermo Fisher Scientific, Waltham, Mass.) or Poznan medium (46) in an anaerobic chamber (Model AS-580, Anaerobe Systems, Morgan, Hill, Calif.). When appropriate, antibiotics were added to the medium at the following final concentrations: 5 μg/ml chloramphenicol and/or 1 mg/ml hygromycin B for P. UF1 isogenic strains; 100 μg/ml ampicillin for E. coli isogenic strains.

For deleting cobA gene from P. UF1, a 644-bp internal fragment of cobA was PCR amplified from P. UF1 chromosome using primers cobA bam-F and cobA xba-R. The purified fragment was cloned into pUCC plasmid (25), generating suicide plasmid pUCC-cobA. Following electroporation into P. UF1 (25), the chloramphenicol resistant colonies were selected and the ΔcobA P. UF1 mutant was identified by PCR analysis using primers P1 and P2. For complementation of the cobA knockout bacterial strain, the cobA gene with the native promoter was integrated into ΔcobA P. UF1 chromosome at the amyE locus, and the resultant C-ΔcobA P. UF1 strain was identified by PCR analysis using primers P3 and P4.

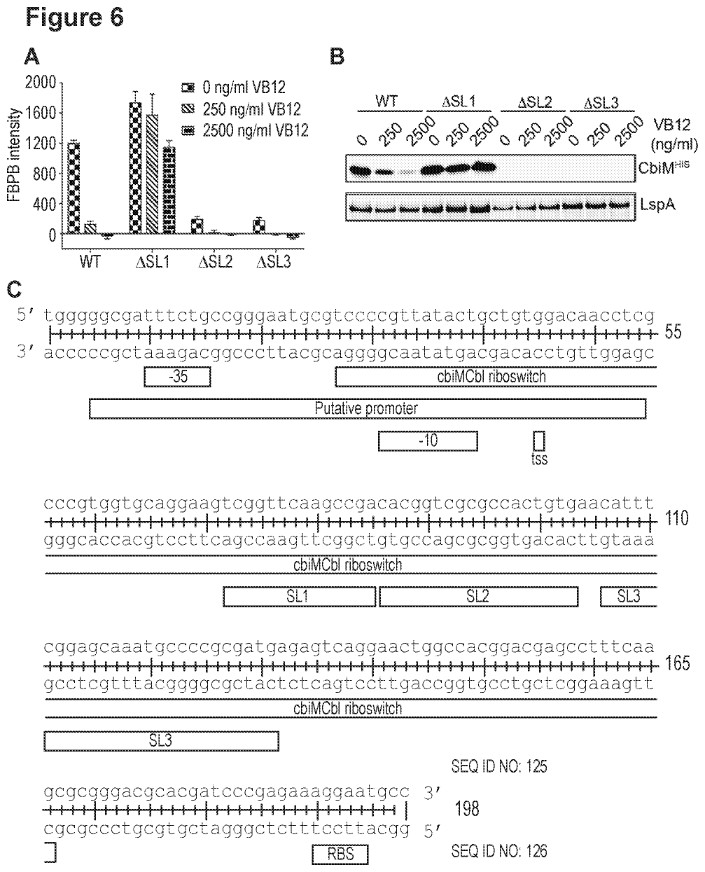

To generate the cbiMCbl riboswitch-FbFP translational fusion constructs, the 5′ UTR (FIG. 6C) containing the promoter of cobA operon, wild-type cbiMCbl riboswitch and ribosome-binding site was PCR amplified from P. UF1 genome (NCBI Genome database, CP018002) with primers S-cobA-1 and S-cobA 22. The codon-optimized FbFP gene (32) was synthesized (GenScript, Piscataway, N.J.) and PCR amplified using primers S-cobA 33 and S-cobA 4. Overlapping PCR was used to ligate the 5′ UTR and FbFP gene using Primers cobA-1 and S-cobA-4, which was subsequently cloned into plasmid pYMZ between BamHI and XbaI sites. Mutations of the cbiMCbl riboswitch were generated by site-directed mutagenesis or overlapping PCR (Table S1). cbiM, cbiN, cbiO, cobA, and tetR reporters were constructed using similar strategies. All plasmids were verified via Sanger sequencing (Genewiz, South Plainfield, N.J.) and introduced into P. UF1 or ΔcobA P. UF1 by electroporation.

For overexpressing cobA operon in ΔcobA P. UF1, the cobA operon with the 5′ UTR was PCR amplified from P. UF1 genomic DNA with primers cbimop-sbf-F and cbimop-hid-R. After digestion with SbfI and HindIII, the purified PCR products were cloned into pYMZ vector, and the resulting construct was electroporated into ΔcobA P. UF1 to obtain OW-operon strain. To investigate the effect of SL1 deletion on VB12 biosynthesis, the SL1 region was deleted from 5′ UTR and the resulting fragment, along with the cobA operon, was ligated by overlapping PCR using primers cbimop-sbf-F/ΔSL1-R and ΔSL1-Fcbimop-hid-R. The constructed plasmid was electroporated into ΔcobA P. UF1 to obtain OΔSL1-operon strain. All obtained strains are listed in Table 1.

| Plasmids/strains | Description |
|---|---|
| Plasmids | |
| pYMZ | Expressing vector in P. UF1, hygB^{R}. |
| pUCC | Vector used for chromosomal insertion in P. UF1, derived of pUC19, |
| | cm^{R}. |
| pUCH | Vector used for complementary expression in P. UF1, derived of |
| | pUC19, hygB^{R}. |
| pET21b | Vector used for protein overexpression in E. coli, Amp^{R} |
| Strains | |
| NEB ® 5-alpha | Competent E. coli used for gene cloning. |
| Rosetta E. coli | Protein overexpression |
| E-21 CobA | Rosetta E. coli overexpressing CobA protein |
| P. UF1 | Propionibacterium UF1 was isolated from human breast milk. |
| | Deletion of cobA gene in P. UF1 using pUCC |
| ΔcobA P. UF1 | Complementary expression of cobA gene in ΔcobA P. UF1 by the |
| C-ΔcobA P. UF1 | native promoter of cobA using pUCH. |
| FbFP-WT | Transforming pYMZ harboring cbiMCbl riboswitch and ΔFbFP into |
| | P. UF1. |
| CbiM-WT | Transforming pYMZ harboring cbiMCbl riboswitch and cbiM^{HIS6} into |
| | P. UF1. |
| FbFP-ΔcobA | Transforming pYMZ harboring cbiMCbl riboswitch and FbFP into |
| | ΔcobA P. UF1. |
| CbiM-ΔcobA | Transforming pYMZ harboring cbiMCbl riboswitch and cbiM^{HIS6} into |
| | ΔcobA P. UF1. |
| CbiN-ΔcobA | Transforming pYMZ harboring cbiMCbl riboswitch and cbiN^{HIS6} into |
| | ΔcobA P. UF1. |
| CbiO-ΔcobA | Transforming pYMZ harboring cbiMCbl riboswitch and cbiO^{HIS6} into |
| | ΔcobA P. UF1. |
| CobA-ΔcobA | Transforming pYMZ harboring cbiMCbl riboswitch and cobA^{HIS6} into |
| | ΔcobA P. UF1. |
| DSL1F-ΔcobA | Transforming pYMZ harboring cbiMCbl riboswitch deleting SL1 |
| | mutant and FibFP into ΔcobA P. UF1. |
| DSL1CM ΔcobA | Transforming pYMZ harboring cbiMCbl riboswitch deleting SL1 |
| | mutant and cbiM^{HIS6} into ΔcobA P. UF1. |
| DSL2F ΔcobA | Transforming pYMZ harboring cbiMCbl riboswitch deleting SL2 |
| | mutant and FbFP into ΔcobA P. UF1. |
| DSL2CM ΔcobA | Transforming pYMZ harboring ccbiMCbl riboswitch deleting SL2 |
| | mutant and cbiM^{HIS6} into ΔcobA P. UF1. |
| DSL3F ΔcobA | Transforming pYMZ harboring cbiMCbl riboswitch deleting SL3 |
| | mutant and FbFP into ΔcobA P. UF1. |
| DSL3CM ΔcobA | Transforming pYMZ harboring cbiMCbl riboswitch deleting SL3 |
| | mutant and cbiM^{HIS6} into ΔcobA P. UF1. |
| D5-6F ΔcobA | Transforming pYMZ harboring cbiMCbl riboswitch D5-6 mutant and |
| | FbFP into ΔcobA P. UF1. |
| D5-6CM ΔcobA | Transforming pYMZ harboring cbiMCbl riboswitch D5-6 mutant and |
| | cbiM^{HIS6} into ΔcobA P. UF1. |
| D7-8FΔcobA | Transforming pYMZ harboring cbiMCbl riboswitch D7-8 mutant and |
| | FbFP into ΔcobA P. UF1. |
| D7-8CM ΔcobA | Transforming pYMZ harboring cbiMCbl riboswitch D7-8 mutant and |
| | cbiM^{HIS6} into ΔcobA P. UF1. |
| D9-10F ΔcobA | Transforming pYMZ harboring cbiMCbl riboswitch D9-10 mutant |
| | and FbFP into ΔcobA P. UF1. |
| D9-10CM ΔcobA | Transforming pYMZ harboring cbiMCbl riboswitch D9-10 mutant |
| | and cbiM^{HIS6} into ΔcobA P. UF1. |
| D11-12F ΔcobA | Transforming pYMZ harboring cbiMCbl riboswitch D11-12 mutant |
| | and FbFP into ΔcobA P. UF1. |
| D11-12CM ΔcobA | Transforming pYMZ harboring cbiMCbl riboswitch D11-12 mutant |
| | and cbiM^{HIS6} into ΔcobA P. UF1. |
| D13-14F ΔcobA | Transforming pYMZ harboring cbiMCbl riboswitch D13-14 mutant |
| | and FbFP into ΔcobA P. UF1. |
| D13-14CM ΔcobA | Transforming pYMZ harboring cbiMCbl riboswitch D13-14 mutant |
| | and cbiM^{HIS6} into ΔcobA P. UF1. |
| D11-14F ΔcobA | Transforming pYMZ harboring cbiMCbl riboswitch D11-14 mutant |
| | and FbFP into ΔcobA P. UF1. |
| D11-14CM ΔcobA | Transforming pYMZ harboring cbiMCbl riboswitch D11-14 mutant |
| | and cbiM^{HIS6} into ΔcobA P. UF1. |
| D11F ΔcobA | Transforming pYMZ harboring cbiMCbl riboswitch D11 mutant and |
| | FbFP into ΔcobA P. UF1. |
| D11CM ΔcobA | Transforming pYMZ harboring cbiMCbl riboswitch D11 mutant and |
| | cbiM^{HIS6} into ΔcobA P. UF1. |
| D12F ΔcobA | Transforming pYMZ harboring cbiMCbl riboswitch D12 mutant and |
| | FbFP into ΔcobA P. UF1. |
| D12CM ΔcobA | Transforming pYMZ harboring cbiMCbl riboswitch D12 mutant and |
| | cbiM^{HIS6} into ΔcobA P. UF1. |
| D13F ΔcobA | Transforming pYMZ harboring cbiMCbl riboswitch D13 mutant and |
| | FbFP into AcobA P. UF1. |
| D13CM ΔcobA | Transforming pYMZ harboring cbiMCbl riboswitch D13 mutant and |
| | cbiM^{HIS6} into ΔcobA P. UF1. |
| D14F ΔcobA | Transforming pYMZ harboring cbiMCbl riboswitch D14mutant and |
| | FbFP into ΔcobA P. UF1. |
| D14CM ΔcobA | Transforming pYMZ harboring cbiMCbl riboswitch D13 mutant and |
| | cbiM^{HIS6} into ΔcobA P. UF1. |
| D12′-15′F ΔcobA | Transforming pYMZ harboring cbiMCbl riboswitch D12′-15′ mutant |
| | and FbFP into ΔcobA P. UF1. |
| D12′-15′CM ΔcobA | Transforming pYMZ harboring cbiMCbl riboswitch D12′-15′ mutant |
| | and cbiM^{HIS6} into ΔcobA P. UF1. |
| D12′-13′F ΔcobA | Transforming pYMZ harboring cbiMCbl riboswitch D12′-13′ mutant |
| | and FbFP into ΔcobA P. UF1. |
| D12′-13′CM ΔcobA | Transforming pYMZ harboring cbiMCbl riboswitch D12′-13′ mutant |
| | and cbiM^{HIS6} into ΔcobA P. UF1. |
| D4′-5′F ΔcobA | Transforming pYMZ harboring cbiMCbl riboswitch D4′-5′ mutant |
| | and FbFP into ΔcobA P. UF1. |
| D4′-5′CM ΔcobA | Transforming pYMZ harboring cbiMCbl riboswitch D4′-5′ mutant |
| | and cbiM^{HIS6} into ΔcobA P. UF1. |
| D14′F ΔcobA | Transforming pYMZ harboring cbiMCbl riboswitch D14′ mutant and |
| | FbFP into ΔcobA P. UF1. |
| D14′CM ΔcobA | Transforming pYMZ harboring cbiMCbl riboswitch D14′ mutant and |
| | cbiM^{HIS6} into ΔcobA P. UF1. |
| D15′F ΔcobA | Transforming pYMZ harboring cbiMCbl riboswitch D15′ mutant and |
| | FbFP into ΔcobA P. UF1. |
| D15′CM ΔcobA | Transforming pYMZ harboring cbiMCbl riboswitch D15′ mutant and |
| | cbiM^{HIS6} into ΔcobA P. UF1. |
| N1F ΔcobA | Transforming pYMZ harboring cbiMCbl riboswitch N1 mutant and |
| | FbFP into ΔcobA P. UF1. |
| N1CM ΔcobA | Transforming pYMZ harboring cbiMCbl riboswitch N1 mutant and |
| | cbiM^{HIS6} into ΔcobA P. UF1. |
| N2F ΔcobA | Transforming pYMZ harboring cbiMCbl riboswitch N2 mutant and |
| | FbFP into ΔcobA P. UF1. |
| N2CM ΔcobA | Transforming pYMZ harboring cbiMCbl riboswitch N2 mutant and |
| | cbiM^{HIS6} into ΔcobA P. UF1. |
| N3F ΔcobA | Transforming pYMZ harboring cbiMCbl riboswitch N3 mutant and |
| | FbFP into ΔcobA P. UF1. |
| N3CM ΔcobA | Transforming pYMZ harboring cbiMCbl riboswitch N3 mutant and |
| | cbiM^{HIS6} into ΔcobA P. UF1. |
| N4F ΔcobA | Transforming pYMZ harboring cbiMCbl riboswitch N4mutant and |
| | FbFP into ΔcobA P. UF1. |
| N4CM ΔcobA | Transforming pYMZ harboring cbiMCbl riboswitch N4 mutant and |
| | cbiM^{HIS6} into ΔcobA P. UF1. |
| N5F ΔcobA | Transforming pYMZ harboring cbiMCbl riboswitch N5 mutant and |
| | FbFP into ΔcobA P. UF1. |
| N5CM ΔcobA | Transforming pYMZ harboring cbiMCbl riboswitch N5 mutant and |
| | cbiM^{HIS6} into ΔcobA P. UF1. |
| AN1F ΔcobA | Transforming pYMZ harboring cbiMCbl riboswitch AN1mutant and |
| | FbFP into ΔcobA P. UF1. |
| AN1CM ΔcobA | Transforming pYMZ harboring cbiMCbl riboswitch AN1 mutant and |
| | cbiM^{HIS6} into ΔcobA P. UF1. |
| AN2F ΔcobA | Transforming pYMZ harboring cbiMCbl riboswitch AN2 mutant and |
| | FbFP into ΔcobA P. UF1. |
| AN2CM ΔcobA | Transforming pYMZ harboring cbiMCbl riboswitch AN2 mutant and |
| | cbiM^{HIS6} into ΔcobA P. UF1. |
| AN3F ΔcobA | Transforming pYMZ harboring cbiMCbl riboswitch AN3 mutant and |
| | FbFP into ΔcobA P. UF1. |
| AN3CM ΔcobA | Transforming pYMZ harboring cbiMCbl riboswitch AN3 mutant and |
| | cbiM^{HIS6} into ΔcobA P. UF1. |
| AN4F ΔcobA | Transforming pYMZ harboring cbiMCbl riboswitch AN4mutant and |
| | FbFP into ΔcobA P. UF1. |
| AN4CM Δcob | Transforming pYMZ harboring cbiMCbl riboswitch AN4 mutant and |
| | cbiM^{HIS6} into ΔcobA P. UF1. |
| AN5FΔcobA | Transforming pYMZ harboring cbiMCbl riboswitch AN5 mutant and |
| | FbFP into ΔcobA P. UF1. |
| AN5CM ΔcobA | Transforming pYMZ harboring cbiMCbl riboswitch AN5 mutant and |
| | cbiM^{HIS6} into ΔcobA P. UF1. |
| | Transforming pYMZ harboring cbiMCbl riboswitch N: AN1mutant |
| | and FbFP into ΔcobA P. UF1. |
| N: AN1F ΔcobA | Transforming pYMZ harboring cbiMCbl riboswitch N: AN1 mutant |
| | and cbiM^{HIS6} into ΔcobA P. UF1. |
| N: AN1CM ΔcobA | Transforming pYMZ harboring cbiMCbl riboswitch N: AN2 mutant |
| | and FbFP into ΔcobA P. UF1. |
| N: AN2F ΔcobA | Transforming pYMZ harboring cbiMCbl riboswitch N: AN2 mutant |
| | and cbiM^{HIS6} into ΔcobA P. UF1. |
| N: AN2CM ΔcobA | Transforming pYMZ harboring cbiMCbl riboswitch N: AN3 mutant |
| | and FbFP into ΔcobA P. UF1. |
| N: AN3F ΔcobA | Transforming pYMZ harboring cbiMCbl riboswitch N: AN3 mutant |
| | and cbiM^{HIS6} into ΔcobA P. UF1. |
| N: AN3CM ΔcobA | Transforming pYMZ harboring cbiMCbl riboswitch N: AN4mutant |
| | and FbFP into ΔcobA P. UF1. |
| N: AN4F ΔcobA | Transforming pYMZ harboring cbiMCbl riboswitch N: AN4 mutant |
| | and cbiM^{HIS6} into ΔcobA P. UF1. |
| N: AN4CM ΔcobA | Transforming pYMZ harboring cbiMCbl riboswitch N: AN5 mutant |
| | and FbFP into ΔcobA P. UF1. |
| N: AN5F ΔcobA | |
| N: AN5CM ΔcobA | Transforming pYMZ harboring cbiMCbl riboswitch N: AN5 mutant |
| | and cbiM^{HIS6} into ΔcobA P. UF1. |
| R1F ΔcobA | Transforming pYMZ harboring cbiMCbl riboswitch R1mutant and |
| | FbFP into ΔcobA P. UF1. |
| R1CM ΔcobA | Transforming pYMZ harboring cbiMCbl riboswitch R1 mutant and |
| | cbiM^{HIS6} into ΔcobA P. UF1. |
| R2F ΔcobA | Transforming pYMZ harboring cbiMCbl riboswitch R2 mutant and |
| | FbFP into ΔcobA P. UF1. |
| R2CM ΔcobA | Transforming pYMZ harboring cbiMCbl riboswitch R2 mutant and |
| | cbiM^{HIS6} into ΔcobA P. UF1. |
| R3F ΔcobA | Transforming pYMZ harboring cbiMCbl riboswitch R3 mutant and |
| | FbFP into ΔcobA P. UF1. |
| R3CM ΔcobA | Transforming pYMZ harboring cbiMCbl riboswitch R3 mutant and |
| | cbiM^{HIS6} into ΔcobA P. UF1. |
| AR1F ΔcobA | Transforming pYMZ harboring cbiMCbl riboswitch AR1mutant and |
| | FbFP into ΔcobA P. UF1. |
| AR1CM ΔcobA | Transforming pYMZ harboring cbiMCbl riboswitch AR1 mutant and |
| | cbiM^{HIS6} into ΔcobA P. UF1. |
| AR2F ΔcobA | Transforming pYMZ harboring cbiMCbl riboswitch AR2 mutant and |
| | FbFP into ΔcobA P. UF1. |
| AR2CM ΔcobA | Transforming pYMZ harboring cbiMCbl riboswitch AR2 mutant and |
| | cbiM^{HIS6} into ΔcobA P. UF1. |
| AR3F ΔcobA | Transforming pYMZ harboring cbiMCbl riboswitch AR3 mutant and |
| | FbFP into ΔcobA P. UF1. |
| AR3CM ΔcobA | Transforming pYMZ harboring cbiMCbl riboswitch AR3 mutant and |
| | cbiM^{HIS6} into ΔcobA P. UF1. |
| R: AR1F ΔcobA | Transforming pYMZ harboring cbiMCbl riboswitch R: AR1mutant |
| | and FbFP into ΔcobA P. UF1. |
| R: AR1CM ΔcobA | Transforming pYMZ harboring cbiMCbl riboswitch R: AR1 mutant |
| | and cbiM^{HIS6}6 into ΔcobA P. UF1. |
| | Transforming pYMZ harboring cbiMCbl riboswitch R: AR2 mutant |
| | and FbFP into ΔcobA P. UF1. |
| R: AR2F ΔcobA | Transforming pYMZ harboring cbiMCbl riboswitch R:AR2 mutant |
| | and cbiM^{HIS6} into ΔcobA P. UF1. |
| R: AR2CM ΔcobA | Transforming pYMZ harboring cbiMCbl riboswitch R: AR3 mutant |
| | and FbFP into ΔcobA P. UF1. |
| R: AR3F ΔcobA | Transforming pYMZ harboring cbiMCbl riboswitch R: AR3 mutant |
| | and cbiM^{HIS6} into ΔcobA P. UF1. |
| R: AR3CM ΔcobA | Transforming pYMZ harboring cbiMCbl riboswitch R: AR3 mutant |
| | and cbiO^{HIS6} into ΔcobA P. UF1. |
| R: AR3CO ΔcobA | Transforming pYMZ harboring cbiMCbl riboswitch and tetR into |
| | ΔcobAP. UF1. |
| R: tR ΔcobA | Transforming pYMZ harboring cbiMCbl riboswitch R: AR3 mutant |
| | and tetR into ΔcobA P. UF1. |
| R: AR3tR ΔcobA | Transforming pYMZ harboring cbiMCbl riboswitch and cobA operon |
| | into ΔcobA P. UF1. |
| OW-operon ΔcobA | Transforming pYMZ harboring cbiMCbl riboswitch deleting SL1 |
| | mutant and cobA operon into ΔcobA P. UF1. |
| ODSL1-operon ΔcobA | |

Vitamin B12 extraction and analysis: Bacterial cultures were centrifuged at 15,000 g for 10 minutes at 4° C. and washed twice using PBS. The cells were disrupted by boiling for 15 minutes in 0.1 M phosphate buffer containing 0.01% potassium cyanide at pH 6.0. After centrifugation at 15,000 g for 2 min, the supernatants were collected and passed through 0.22 μm filters (Millipore, Burlington, Mass.). Vitamin B12 (VB12) in the filtrates was quantified by Agilent 1220 infinity II LC system comprised of an automated sampler (G4282B), gradient Pump (G4281 B) and VWD detector (G4284B). Separations were performed using the following mobile phase (46), 0.25 M NaH₂PO₄, pH 3.5 (phosphoric acid): methanol (75:25) in an Agilent C18 column (Eclipse Plus C18, 3.5 μm, 4.6×150 mm column) at 20° C. with a flow rate of 1.0 ml/min. The total HPLC run time for each sample was 15 minutes, and the injection volume was 20 μl. The detector wavelength was set at 362 nm. Quantitation was based on peak area and the standard curve of VB12. Data acquisition and analysis were done using Agilent ChemStation.

RNA Sequencing: For bacterial RNA-seq, total RNA was isolated from P. UF1 and ΔcobA P. UF1 strains and the DNA libraries were constructed essentially as previously described (25). The obtained cDNA libraries were sequenced on an Illumina HiSeq instrument (Illumina, Inc., San Diego, Calif.) at the University of Florida ICBR NextGen DNA Sequencing Core Facility. Sequence analyses of bacterial samples were performed as described previously (25), with some modifications. Principal component analysis was performed upon regularized-log-transformation of the count data using Python Scikit-learn package. Pathway analysis was analyzed using an in-house informatics pipeline, which modified from Mummichog's pathway analysis module (47). Briefly, the Enzyme Commission number (EC number) of each annotated gene of P. UF1 genome was extracted from databases including KEGG and BioCyc. The genes were categorized into pathways and modules from KEGG database based on the EC number. 71 significantly upregulated and 73 downregulated genes in P. UF1 compared to ΔcobA P. UF1 were used as input for pathway analysis (p<0.05, fold change >2). Pathway enrichment p-values were calculated based on permutation in Mummichog's pathway analysis module. The pathways represented by at least two genes and enriched at p<0.05 are presented. Heatmap analysis was generated on selected significant genes (p<0.05, fold change >1.5) related to the indicated metabolic pathways.

High-resolution metabolomics analysis: Bacterial samples were processed and metabolites were extracted as previously described (25). Adaptive processing of Liquid Chromatography-Mass Spectrometry (apLCMS) was used to perform peak detection, noise filtering, mass to charge (m/z) ratio and retention time alignment and feature quantification. In-house informatics pipelines were used to quality control and clean up the feature table. PCA was performed on log 2-transformed intensity values of all metabolite features. Subsequently, student's t-test was used to evaluate difference of metabolite features between P. UF1 and ΔcobA P. UF1. False discovery rate (FDR) was calculated using the Benjamini-Hochberg method. FDR-adjusted p-values less than 0.05 were considered significant. Metabolic pathway analysis was performed by Mummichog (v1.0.5) (47), with a modified adduct function to include M+H—CN[2+], an experiment-verified metabolite adduct that matches VB12. 283 significant metabolite features were used as input to Mummichog (FDR adjusted p<0.05, student's t-test). The total list of features was used as reference. Pathway enrichment p-values were calculated in Mummichog based on permutation. The pathways represented by at least two metabolite features and enriched at p<0.05 are presented. KEGG database was used to identify metabolite features related to significant pathways identified by Mummichog. Identified metabolite features with adduct ions of M+H[1+] or M+H—CN[2+], were selected to show in the scatter plot, with each dot representing the log 2-transformed intensity values.

FbFP reporter assays: To perform FbFP fluorescence intensity measurement, bacteria were cultivated for three days in triplicate, washed, and resuspended with PBS. The excitation and emission wavelengths of FbFP were set at 452 nm and 495 nm on a microplate reader (BioTek, Winooski, Vt.), respectively (48). FbFP intensity at 495 nm was normalized by corresponding OD₆₀₀ values for each sample. P. UF1 strain with an empty vector was used as the baseline for calculation of fluorescence intensity.

Preparation of anti-CobA serum antibodies: To generate CobA polyclonal antibodies, the pET21b-cobA expression plasmid was constructed by PCR amplification of the cobA gene using primers 21-cobA-bamF and 21-cobA-xhoR (provided in Table 2).

TABLE 2
Primers

| | | SEQ | |
|---|---|---|---|
| Name | Sequence (5′-3′) | ID NO: | Usage |
| cobAbam-F | CCCGGATCCctcgtcggcgccgggcccggc | 1 | Deletion of cobA gene in P. UF1 |
| cobAxba-R | CCCTCTAGAgeggacgcacgtggtgegegtcca | 2 | For identifying cobA mutant and |
| P1 | gtggcactcgtggtgatctt | 3 | complementary strain |
| P2 | GAGCGAGGAAGCGGAAGAG | 4 | |
| P3 | ccccggcgcctacaaggacatcga | 5 | For constructing strain for |
| P4 | ccgccctcctgttgatgttctggt | 6 | complementary expression of cobA |
| Amy-cobA-1 | CCCGGATCCtcagacgccgcggcgtccaccc | 7 | gene in ΔcobA P. |
| Amy-cobA-2 | cgtggggccgtcgtagtgggacgatgcatgtgagcgactggcgacgaga | 8 | |
| Amy-cobA-3 | tctcgtcgccagtcgctcacatgcatcgtcccactacgacggccccacg | 9 | |
| Amy-cobA-4 | gccgggcaacagtgtggtggtcatgacgcacgatcccgagaaaggaatgcc | 10 | |
| Amy-cobA-5 | ggcattcctttctcgggatcgtgcgtcatgaccaccacactgttgcccggc | 11 | |
| Amy-cobA-6 | CCCTCTAGAtcagtggtcgctgggcgcgcgat | 12 | |
| 21-cobA-F | CCCGGATCCatgaccaccacactgttgcccggca | 13 | Overexpression of cobA in E.coli |
| 21-cobA-R | GCGCTCGAGgtggtcgctgggcgcgcgatgg | 14 | |
| S-cobA-1 | CCCGGATCCcgtcccactacgacggccc | 15 | For constructing FbFP-WT |
| | | | and CbiM-WT mutants |
| S-cobA-22 | TGGTGGTGCGACGAGCCCATggcattcctttctcgggatcgtg | 16 | For constructing FbFP-WT mutant |
| S-cobA-33 | cacgatcccgagaaaggaatgccATGGGCTCGTCGCACCACCA | 17 | For constructing FbFP-WT mutant |
| S-cobA-4 | CCCTCTAGATCAGTGCTTGGCCTGGCCCT | 18 | |
| ScbiM-5 | CCCTCTAGAtcaGTGGTGGTGGTGGTGGTGgacatgtgccacctccggaa | 19 | For constructing W-CM mutant |
| | cct | | |
| DSL1-F | gttcacagtggcgcgaccgtg cttcctgcaccacgggcgagg | 20 | For constructing DSL1F/CM |
| DSL1-R | cctcgcccgtggtgcaggaag cacggtcgcgccactgtgaac | 21 | mutants |
| DSL2-F | ggggcatttgctccgaaatgtt tcggcttgaaccgacttcctgcac | 22 | For constructing DSL2F/CM |
| DSL2-R | gtgcaggaagtcggttcaagccga aacatttcggagcaaatgcccc | 23 | mutants |
| DSL3-F | gtccgtggccagttcctgactct gttcacagtggcgcgaccgtg | 24 | For constructing DSL3F/CM |
| DSL3-R | cacggtcgcgccactgtgaac agagtcaggaactggccacggac | 25 | mutants |
| SL1-5-6F | tcgcccgtggtgcaggaagtcggGCcaagccgacacggtcgcgccactgtgaacatttc | 26 | For constructing D5-6F/CM |
| SL1-5-6R | gaaatgttcacagtggcgcgaccgtgtcggcttgGCccgacttcctgcaccacgggcga | 27 | mutants |
| SL1-7-8F | TcgcccgtggtgcaggaagtcggttTGagccgacacggtcgcgccactgtgaacatttc | 28 | For constructing D7-8F/CM |
| SL1-7-8R | gaaatgttcacagtggcgcgaccgtgtcggctCAaaccgacttcctgcaccacgggcgA | 29 | mutants |
| SL1-9-10F | TcgcccgtggtgcaggaagtcggttcaCTccgacacggtcgcgccactgtgaacatttc | 30 | For constructing D9-10F/CM |
| SL1-9-10R | gaaatgttcacagtggcgcgaccgtgtcggAGtgaaccgacttcctgcaccacgggcgA | 31 | mutants |
| SL1-11-12F | TcgcccgtggtgcaggaagtcggttcaagTAgacacggtcgcgccactgtgaacatttc | 32 | For constructing D11-12F/CM |
| SL1-11-12R | gaaatgttcacagtggcgcgaccgtgtcTActtgaaccgacttcctgcaccacgggcgA | 33 | mutants |
| SL1-13-14F | TcgcccgtggtgcaggaagtcggttcaagccTGcacggtcgcgccactgtgaacatttc | 34 | For constructing D13-14F/CM |
| SL1-13-14R | gaaatgttcacagtggcgcgaccgtgCAggcttgaaccgacttcctgcaccacgggcgA | 35 | mutants |
| SL1-11-14F | tcgcccgtggtgcaggaagtcggttcaagATAGcacggtcgcgccactgtgaacatttc | 36 | For constructing D11-14F/CM |
| SL1-11-14R | gaaatgttcacagtggcgcgaccgtgCTATcttgaaccgacttcctgcaccacgggcga | 37 | mutants |
| A9G-F | tcgcccgtggtgcaggaagtcggttca GgccgacacggtcgcgccactgtgaaCA | 38 | For constructing D9F/CM mutants |
| A9G-R | TGttcacagtggcgcgaccgtgtcggcCtgaaccgacttcctgcaccacgggcga | 39 | |
| C11A-F | gcccgtggtgcaggaagtcggttcaagAcgacacggtcgcgccactgtgaacatt | 40 | For constructing D11F/CM mutants |
| C11A-R | aatgttcacagtggcgcgaccgtgtcgTcttgaaccgacttcctgcaccacgggc | 41 | |
| C12A-F | gcccgtggtgcaggaagtcggttcaagcAgacacggtcgcgccactgtgaacatt | 42 | For constructing D12F/CM mutants |
| C12A-R | aatgttcacagtggcgcgaccgtgtcTgcttgaaccgacttcctgcaccacgggc | 43 | |
| G10T-F | tcgcccgtggtgcaggaagtcggttca aTccgacacggtcgcgccactgtgaaCA | 44 | For constructing D10F/CM mutants |
| G10T-R | TGttcacagtggcgcgaccgtgtcggAttgaaccgacttcctgcaccacgggcga | 45 | |
| G13T-F | cccgtggtgcaggaagtcggttcaagcc Tacacggtcgcgccactgtgaacatttcg | 46 | For constructing D13′F/CM mutants |
| G13T-R | cgaaatgttcacagtggcgcgaccgtgtAggcttgaaccgacttcctgcaccacggg | 47 | |
| A14G-F | cccgtggtgcaggaagtcggttcaagcc gGcacggtcgcgccactgtgaacatttcg | 48 | For constructing D14F/CM mutants |
| A14G-R | cgaaatgttcacagtggcgcgaccgtgCcggcttgaaccgacttcctgcaccacggg | 49 | |
| SL3-DF | gttcctgactctcatcgcggggcgctccgaaatgttcacagtggcgc | 50 | For constructing D12′-15′F/CM |
| SL3-DR | gcgccactgtgaacatttcggagc gccccgcgatgagagtcaggaac | 51 | mutants |
| SL3-LSF | cgcgccactgtgaacatttcggagcGGatgccccgcgatgagagtcaggaa | 52 | For constructing D12′-13′F/CM |
| SL3-LSR | ttcctgactctcatcgcggggcatCCgctccgaaatgttcacagtggcgcg | 53 | mutants |
| SL3-SSF | acacggtcgcgccactgtgaacatCCcggagcaaatgccccgcgatga | 54 | For constructing D4′-5′F/CM |
| SL3-SSR | catcgcggggcatttgctccgGGatgttcacagtggcgcgaccgtgt | 55 | mutants |
| 3AGF | tcgcgccactgtgaacatttcggagcaaGtgccccgcgatgagagtcaggaact | 56 | |
| 3AGR | agttcctgactctcatcgcggggcaCttgctccgaaatgttcacagtggcgcga | 57 | |
| 4TCF | tcgcgccactgtgaacatttcggagcaaaCgccccgcgatgagagtcaggaact | 58 | |
| 4TCR | agttcctgactctcatcgcggggcGtttgctccgaaatgttcacagtggcgcga | 59 | For constructing D14′F/CM mutants |
| 2-T-F | gaagtcggttcaagccgacacggt TATAT cactgtgaacatttcggagcaaatgcccc | 60 | |
| 2-T-R | ggggcatttgctccgaaatgttcacagtgATATAaccgtgtcggcttgaaccgacttc | 61 | For constructing D15′F/CM mutants |
| 2-AT-F | aactggccacggacgagcctttcaa ATATAggacgcacgatcccgagaaaggaatg | 62 | |
| 2-AT-R | cattcctttctcgggatcgtgcgtccTATATttgaaaggctcgtccgtggccagtt | 63 | For constructing N3F/CM mutants |
| 3-T-F | gaagtcggttcaagccgacacggt GGGGCcactgtgaacatttcggagcaaatgcccc | 64 | |
| 3-T-R | ggggcatttgctccgaaatgttcacagtgGCCCCaccgtgtcggcttgaaccgacttc | 65 | For constructing N2F/CM mutants |
| 3-AT-F | aactggccacggacgagcctttcaa GCCCCggacgcacgatcccgagaaaggaatg | 66 | |
| 4-AT-R | cattcctttctcgggatcgtgcgtccGGGGCttgaaaggctcgtccgtggccagtt | 67 | For constructing N1F/CM mutants |
| 4-T-F | gaagtcggttcaagccgacacggt GGAGC cactgtgaacatttcggagcaaatgcccc | 68 | |
| 4-T-R | ggggcatttgctccgaaatgttcacagtgGCTCCaccgtgtcggcttgaaccgacttc | 69 | For constructing AN3F/CM and N- |
| 4-AT-F | aactggccacggacgagcctttcaa GCTCCggacgcacgatcccgagaaaggaatg | 70 | AN3F/CM mutants |
| 4-AT-R | cattcctttctcgggatcgtgcgtccGGAGCttgaaaggctcgtccgtggccagtt | 71 | |
| SL2-DLF | aagtcggttcaagccgacacggtcactgtgaacatttcggagcaaatgcc | 72 | For constructing AN2F/CM and N- |
| SL2-DLR | ggcatttgctccgaaatgttcacagtgaccgtgtcggcttgaaccgactt | 73 | AN2F/CM mutants |
| ASL2-DLF | aactggccacggacgagcctttcaaggacgcacgatcccgagaaaggaatg | 74 | For constructing AN1 F/CM and N- |
| ASL2-DLR | cattcctttctcgggatcgtgcgtccttgaaaggctcgtccgtggccagtt | 75 | AN1F/CM mutants |
| SL2-SLF | Aagtcggttcaagccgacacggt gctcgcactgtgaacatttcggagcaaatgcc | 76 | For constructing N5F/CM mutants |
| SL2-SLR | ggcatttgctccgaaatgttcacagtgcgagcaccgtgtcggcttgaaccgactT | 77 | |
| ASL2-SLF | Aactggccacggacgagcctttcaacgagcggacgcacgatcccgagaaaggaatg | 78 | For constructing AN5F/CM and N- |
| ASL2-SLR | cattcctttctcgggatcgtgcgtccgctcgttgaaaggctcgtccgtggccagtT | 79 | AN5F/CM mutants |
| A-site-F | GagtcaggaactggccacggacgagcctCtcaagcgcgggacgcacgatccc | 80 | For constructing N4F/CM mutants |
| A-site-R | gggatcgtgcgtcccgcgcttgaGaggctcgtccgtggccagttcctgactC | 81 | |
| A-region-F | gagtcaggaactggccacggacgagTTCCTtcaagcgcgggacgcacgatccc | 82 | For constructing AN4F/CM and N- |
| A-region-R | gggatcgtgcgtcccgcgcttgaAGGAActcgtccgtggccagttcctgactc | 83 | AN4F/CM mutants |
| R-S-cbimF | aagcgcgggacgcacgatcccgagaGaggaatgccgtgcatatcgcagaaggcgt | 84 | For constructing AR2F/CM and N- |
| R-S-cbimR | acgccttctgcgatatgcacggcattcctCtctcgggatcgtgcgtcccgcgcttg | 85 | AR2F/CM mutants |
| R-R-cbimF | caagcgcgggacgcacgatcccgaAGGAAaatgccgtgcatatcgcagaaggcgt | 86 | For constructing AR3F/CM and N- |
| R-R-cbimR | acgccttctgcgatatgcacggcattTTCCTtcgggatcgtgcgtcccgcgcttg | 87 | AR3F/CM mutants |
| R-S-GFPF | caagcgcgggacgcacgatcccgagaGaggaatgccatgggcTCGTCGCACCAC | 88 | For constructing AR2CM mutant |
| | CA | | |
| R-S-GFPR | TGGTGGTGCGACGAGCCCATggcattcctCtctcgggatcgtgcgtcccgcgctt | 89 | For constructing AR3CM mutant |
| | g | | |
| R-R-GFPF | caagcgcgggacgcacgatcccgaAGGAAaatgccatgggcTCGTCGCACCAC | 90 | |
| | CA | | For constructing AR2F mutant |
| R-R-GFPR | TGGTGGTGCGACGAGCCCATggcattTTCCTtcgggatcgtgcgtcccgcgc | 91 | For constructing AR3F mutant |
| | ttg | | |
| NR-GPF-F | aagcgcgggacgcacgatcccgacttaggaatgccATGGGCTCGTCGCACCAC | 92 | For constructing AR1F mutant |
| | CA | | |
| NR-GPF-R | TGGTGGTGCGACGAGCCCATggcattcctAAGtcgggatcgtgcgtcccgcgc | 93 | For constructing AR1 CM mutant |
| | tt | | |
| NRcbiM-F | aagcgcgggacgcacgatcccgaCTTaggaatgccgtgcatatcgcagaaggcgt | 94 | For constructing AR1F/CM and N- |
| NR-cbiM-R | acgccttctgcgatatgcacggcattcctAAGtcgggatcgtgcgtcccgcgctt | 95 | AR1 F/CM mutants |
| ANR-F | aggaactggccacggacgagcctAAGaagcgcgggacgcacgatcccga | 96 | For constructing CbiO-AcobA |
| ANR-R | tcgggatcgtgcgtcccgcgcttCTTaggctcgtccgtggccagttcct | 97 | |
| w-cbiOF | gacgcacgatcccgagaaaggaatgcCatgagcgccctgctggccgcccac | 98 | For constructing tetR-AcobA |
| w-cbiOR | gtgggcggccagcagggcgctcatGgcattcctttctcgggatcgtgcgtc | 99 | |
| w-tetRF | gacgcacgatcccgagaaaggaatgccatGTCCCGCCTCGACAAGTCCAAG | 100 | |
| | GT | | |
| w-tetRR | ACCTTGGACTTGTCGAGGCGGGACATGgcattcctttctcgggatcgtgcgtc | 101 | |
| tetR-HidR | GGCAAGCTTTTAGGAGCCGGACTCGCACTTGAGCTG | 102 | For constructing R-AR3tR and |
| | | | tetR-WT |
| cbiN-hid | CCCAAGCTTtcaGTGGTGGTggtggtggtgggcgttccgtgatccgggggccgtcg | 103 | For constructing CbiN-AcobA |
| | gtc | | mutant |
| cbiQ-hid | CCCAAGCTTtcaGTGGTGGTGGTGGTggtgtcgggccaccaccaggctgatc | 104 | For constructing CbiQ-AcobA |
| | ga | | mutant |
| cbiO-hid | CCCAAGCTTtcaGTGGTGGTGGTGGTGGtgtcgggtttcctcatcggtgttggt | 105 | For constructing CbiO-AcobA |
| | ggt | | mutant |
| cobA-hid | CCCAAGCTTtcaGTGGTGGTGGTGGTGGTGgtggtcgctgggcgcgcgat | 106 | For constructing CobA-AcobA |
| | ggtc | | mutant |
| m-cbiOF | gacgcacgatcccgagagaggaatgcCatgagcgccctgctggccgcccac | 107 | For constructing R-AR3CO mutant |
| m-cbiOR | gtgggcggccagcagggcgctcatGgcattcctctctcgggatcgtgcgtc | 108 | For constructing R-AR3tR mutant |
| m-tetRF | gacgcacgatcccgagagaggaatgccatGTCCCGCCTCGACAAGTCCAAG | 109 | qPCR |
| | C | | |
| m-tetRR | ACCTTGGACTTGTCGAGGCGGGACATGgcattcctctctcgggatcgtgcgt | 110 | qPCR |
| | c | | |
| GroL2-RT-F | CAATGTCGTGTTGGAGAAG | 111 | qPCR |
| | | | operon/ODSL1-operon mutants |
| GroL2-RT-R | CGCCGATCTTGTGGTAGG | 112 | qPCR |
| qcbiM-F | ctcatcgtgctgatcttcca | 113 | qPCR |
| qcbiM-R | GAGCTTCTTGTTGAGCACATAG | 114 | qPCR |
| qcbiN-F | GGTTCCAGCCGCTGTT | 115 | qPCR |
| qcbiN-R | CCCAGGCAGTAGAAGATGATG | 116 | qPCR |
| qcbiQ-F | CCATCGTGGCTTCGAGAC | 117 | qPCR |
| qcbiQ-R | CCACCAGGCTGATCGAC | 118 | qPCR |
| qcbiQ-F | TGCACCAGATGCGTGAC | 119 | qPCR |
| qcbiQ-R | TCACAGACGATAGCGACCT | 120 | qPCR |
| qcobA-F | CCAGGAGGAGATCAACCAAC | 121 | For constructing OW- |
| qcobA-R | GCCCGAAGACGAACGAG | 122 | operon/ODSL1-operon mutants |
| cbop-hid-R | GGCAAGCTTtcagtggtcgctgggcgcgcgat | 123 | |
| cbop-sbf-F | GCGCCTGCAGGcgtcccactacgacggccccacgg | 124 | |

Following transformation into E. coli Rosetta(DE3), CobA expression was induced with 1 mM isopropyl β-D-1-thiogalactopyranoside (IPTG) (Sigma Aldrich, St. Louis, Mo.). Cell lysates were separated by 12% SDS-PAGE, and the CobA proteins were excised from the gel and used to immunize C57BL/6 mice, resulting in anti-CobA serum antibodies.

Western blot: Bacteria were cultivated for three days, and washed three times, followed by lysozyme digestion (10 mg/ml) for 2 hours at 37° C. Cell lysates were separated by 12% SDS-PAGE, transferred to PVDF membranes (Sigma Aldrich, St. Louis, Mo.), and blocked with Odyssey blocking buffer (Li-Cor Biosciences, Lincoln, Nebr.). Subsequently, membranes were incubated with anti-HIS-tag antibody (Thermo Fisher Scientific, Waltham, Mass.), anti-TetR antibody (Takara Bio, Mountain view, CA), anti-CobA serum antibodies, or anti-LspA serum antibodies (Stock in our lab) for 2 h at room temperature in the blocking buffer with 0.02% Tween 20. After washing with TBST (20 mM Tris, 150 mM NaCl and 0.1% Tween 20, pH 7.4), membranes were incubated with IRDye 680RD goat anti-mouse secondary antibody (Li-Cor Biosciences, Lincoln, Nebr.) for 1 hour at room temperature in the blocking buffer with 0.02% Tween 20. After washing with TBS, the proteins were detected using an Odyssey infrared imaging system (Li-Cor Biosciences, Lincoln, Nebr.). LspA was used as an internal control.

Quantitative RT-PCR: Total RNA was extracted from various bacterial cultures using Trizol reagent (Thermo Fisher Scientific, Waltham, Mass.), and the RNA mixtures were further purified using the Aurum Total RNA Mini Kit (Bio-Rad, Hercules, Calif.). The cDNA was synthesized from total RNA using iScript Advanced cDNA Synthesis Kit (Bio-Rad, Hercules, Calif.). Quantitative real-time was accomplished with SsoAdvanced Universal SYBR Green Supermix (Bio-Rad, Hercules, Calif.) on a CFX96 real-time PCR system (Bio-Rad, Hercules, Calif.) using primers listed in Table 2 above. Results were normalized to those obtained from groL2 gene.

### Example 1—CobA is the Key Enzyme for Bacterial Vitamin B12 (VB12) Biosynthesis

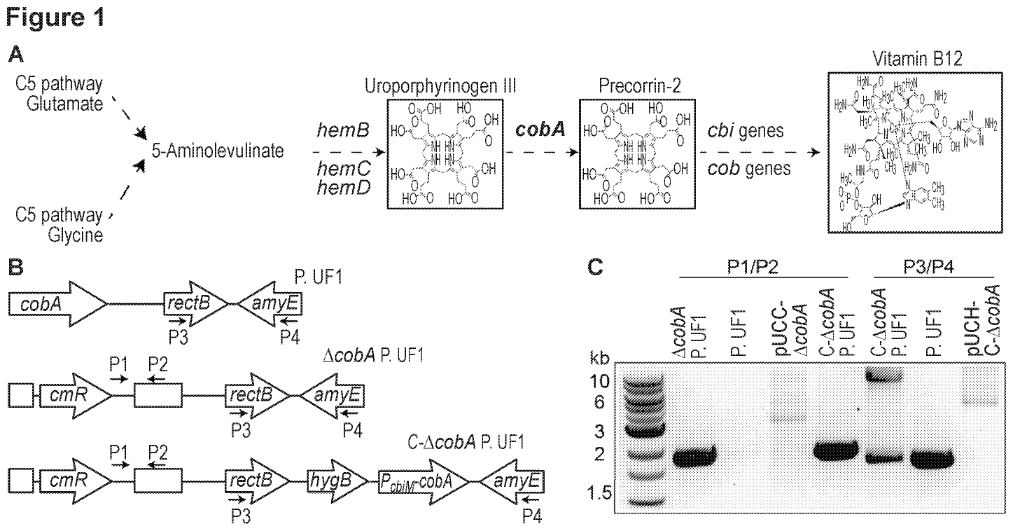

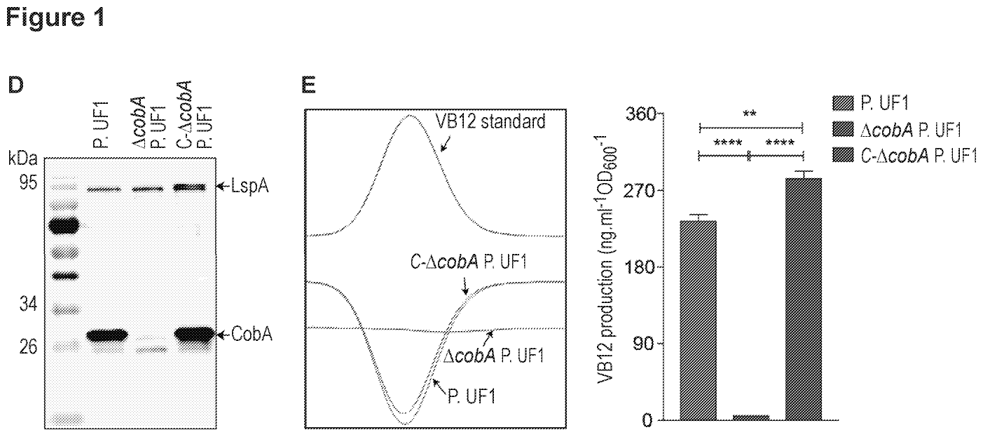

Biochemical studies demonstrated that CobA is essential for the conversion of uroporphyrinogen III to precorrin-2, which is the branch point between the biosynthesis of vitamin B12 (VB12) and siroheme (18) (FIG. 1A). The following Example was performed to determine if CobA plays a critical role in the biosynthesis of VB12 within P. UF1.

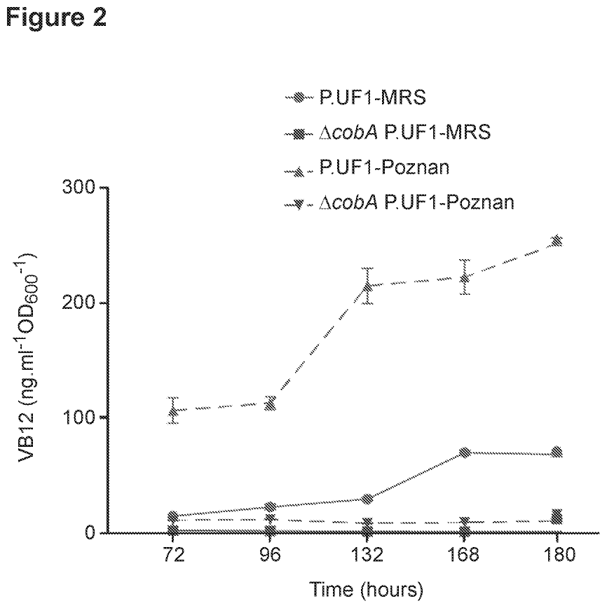

The cobA gene was deleted from the bacterial chromosome by homologous recombination with a single crossover event, resulting in ΔcobA P. UF1 (FIG. 1B and Table 1). For complementation of the cobA-deficient bacterial strain, the cobA gene, along with its native promoter, was integrated into the chromosome of ΔcobA P. UF1 (FIG. 1B and Table 1). PCR and Western blot demonstrated the cobA expression in P. UF1 and C-ΔcobA P. UF1 but not in ΔcobA P. UF1 (FIG. 1C and FIG. 1D). HPLC analysis revealed that cobA deficiency led to complete abrogation of intracellular VB12 within ΔcobA P. UF1, referring to VB12 standard, and the complementation of cobA gene restored VB12 biosynthesis in C-ΔcobA P. UF1 (FIG. 1E). Further, deletion of cobA significantly decreased VB12 production over time when cultured either in MRS, or in Poznan medium, a minimal medium containing no VB12 (FIG. 2). These data highlighted that the pivotal role of CobA that critically directed VB12 biosynthesis within P. UF1.

### Example 2—Transcriptomic and Metabolic Regulation by CobA

Vitamin B12 (VB12), as an enzymatic cofactor, critically regulates the transcriptomic and metabolic machineries in diverse bacteria (29), including human gut microbiota. To underscore the central implication of CobA in regulating gene expression within P. UF1, RNA sequencing (RNA-Seq) was performed (25).

Overall, principal component analysis (PCA) plot exhibited a distinct separation of the global transcriptomes of P. UF1 and ΔcobA P. UF1 (FIG. 2A), demonstrating 561 upregulated and 533 downregulated genes in ΔcobA P. UF1 compared to P. UF1 (data not shown). Henceforth, these differentially expressed genes were used to perform pathway analysis. Obtained data revealed that deletion of cobA mainly led to dysregulation of pathways related to vitamin, amino acid metabolisms and ion transport system (FIG. 2B). Accordingly, genes involved in VB12 (e.g., cbiM, cobS, cobU, cbiF and cobH), VB9 (e.g., metF and moaC2), methionine and cysteine (e.g., cysK-1, cysK-2 and metK) and sulfur metabolisms (e.g., cysP, cysT and cysH) were significantly suppressed in ΔcobA P. UF1 compared to P. UF1 (FIG. 2C).

Further, to investigate whether the observed changes in bacterial transcriptome correlate with metabolomic modifications, high-resolution liquid chromatography-mass spectrometry (LC-MS) was employed to analyze the metabolic profiling of ΔcobA P. UF1 and P. UF1. Here, PCA demonstrated a distinct clustering of the two metabolomes at day 3 and 6 of bacterial cultures (FIG. 2D). Metabolic pathways, including vitamin (e.g., VB12 and VB9), fatty acid (e.g., propanoate) and amino acid (e.g., methionine and cysteine) metabolisms, were significantly altered in ΔcobA P. UF1 compared to P. UF1 (FIG. 2E). Notably, cyanocobalamin, the final product of VB12 biosynthesis, was markedly depleted within ΔcobA P. UF1, whereupon the substrates (e.g., cobalt-precorrin 3 and α-ribazole) were significantly accumulated (FIG. 2F). Together, these findings suggested the critical role of cobA in VB12 biosynthesis regulating transcriptomic and metabolic machineries within P. UF1.

### Example 3—Controlling cobA Operon Expression by VB12

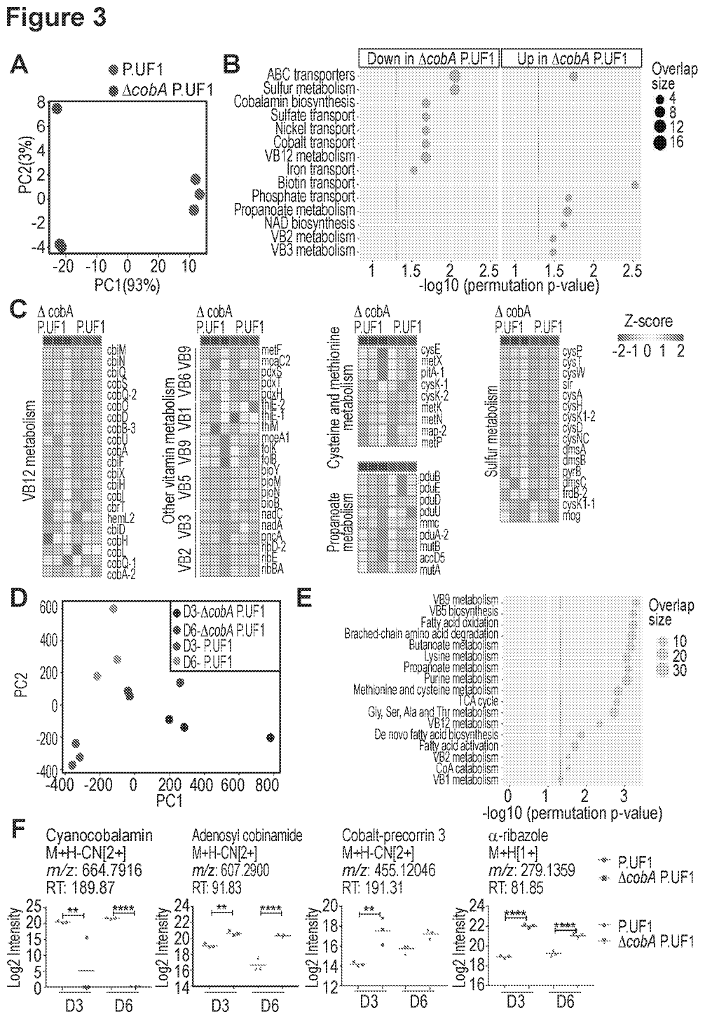

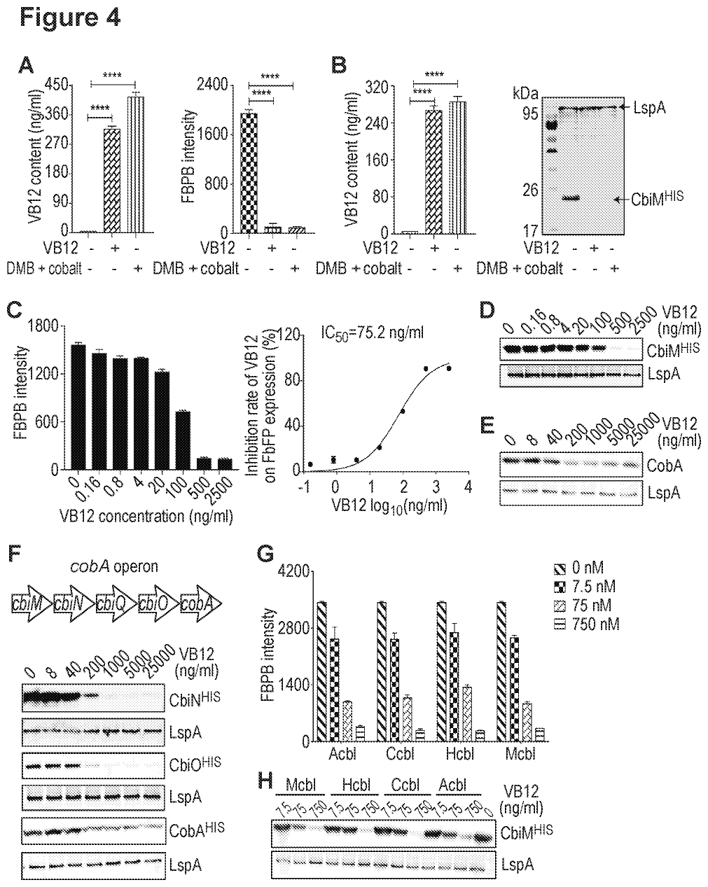

In bacteria such as Escherichia coli (E. col′) and Salmonella typhimurium (S. typhimurium), VB12 interacts with the 5′ UTR of VB12 biosynthesis operon to repress the translation of the corresponding genes, including cob and btuB operon (30, 31). Demonstrating the central role of cobA in controlling VB12 biosynthesis within P. UF1 prompted us to determine the feedback regulation of cobA operon by VB12 (FIG. 1H). Thus, we cloned 5′ UTR of cobA operon (P_{cbiM}) into the first gene of the operon, cbiM, with HIS-tag or flavin mononucleotide-binding fluorescent protein (FbFP) gene (32) to construct the reporter bacterial strains, CbiM-WT and FbFP-WT strains, respectively (Table 1). Here, when adding cobalt and DMB, two substrates required for VB12 biosynthesis, or VB12 alone into FbFP-WT and CbiM-WT bacterial cultures, the expression of FbFP and cbiM was significantly decreased (FIG. 3A and FIG. 3B). To further determine whether there would be a correlation between VB12 concentration and the expression of these reporter genes, the effect of VB12 on FbFP expression was examined. As shown in FIG. 3C, VB12 downregulated the FbFP expression in a VB12 dose-dependent manner. The half maximal inhibitory concentration (IC₅₀) of VB12 was 75.2 ng/ml and the expression of FbFP was completely abated at 500 ng/ml (FIG. 3C). Additionally, Western blot analyses consistently demonstrated the dose-dependent inhibition of cbiM expression by VB12 using HIS-tag antibody (FIG. 3D). Furthermore, the same VB12-mediated gene suppression was also observed in P. UF1 using generated mouse serum antibodies against CobA (FIG. 3E). These data demonstrated that both endogenous and exogenous VB12 modulated the expression of cobA operon through its 5′ UTR.

The cobA operon harbors cbiMNQOA genes encoding proteins critical for cobalt transport and precorrin-2 biosynthesis (FIG. 3F). To investigate whether VB12 regulates the entire cobA operon, the 3′-terminus of respective cbiN, cbiO and cobA genes was labeled with HIS-tag to analyze their expression by Western blot. Results demonstrated the expression of cbiN and cbiO was dampened by adding VB12 to CbiN-ΔcobA and CbiO-ΔcobA bacterial cultures (FIG. 3F and Table 1), as observed for cbiM expression (FIG. 3D). Note that cobA expression displayed the dose-dependent inhibition but to a lesser extent in CobA-ΔcobA strain (FIG. 3F and Table 51). These data suggested that the expression of the entire cobA operon was tightly controlled by VB12.

VB12 possesses analogues such as cyanocobalamin (manufactured form), methylcobalamin (active form), hydroxocobalamin (storage form) and adenosylcobalamin (active form) (14). To elucidate whether there would be a differential regulation by aforementioned VB12 analogues, CbiM-ΔcobA and FbFP-ΔcobA bacterial cultures (Table 1) were treated with various concentrations of the VB12 analogues to analyze the cbiM and FbFP expression. As shown in FIGS. 3G and 3H, the expression of cbiM and FbFP was similarly downregulated by all the analogues. In summary, these data demonstrated VB12 and its analogues controlled the expression of cobA operon through its 5′ UTR.

### Example 4—Identifying a Novel VB12 Riboswitch within 5′ UTR of cobA Operon

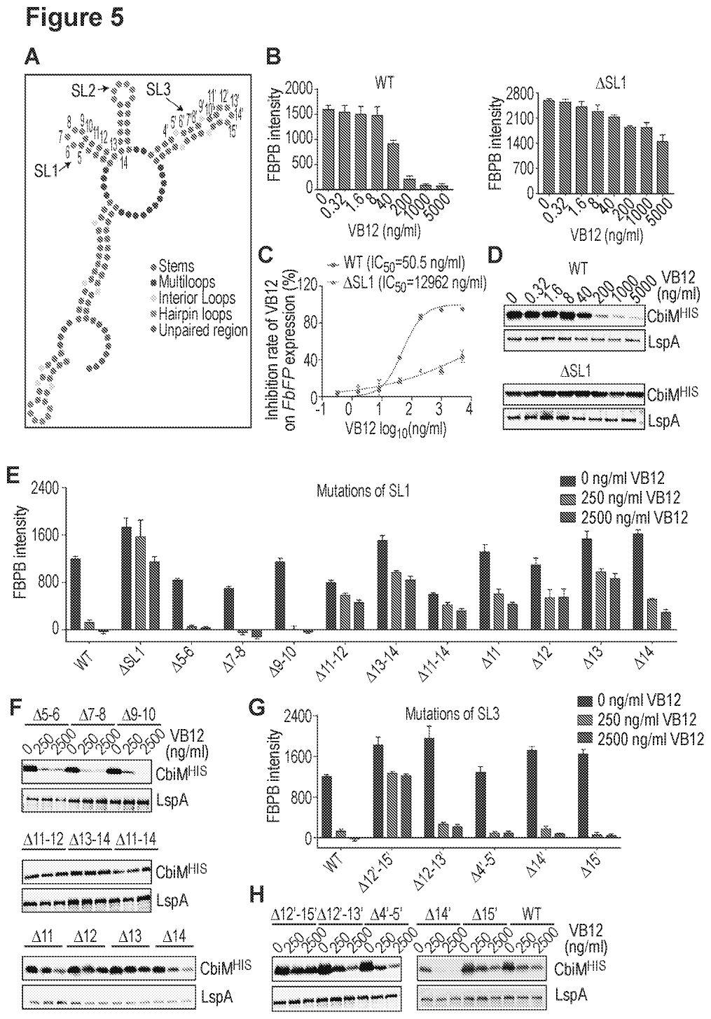

Riboswitches are noncoding RNA (ncRNA) regulatory elements that specifically bind small-molecular ligands such as VB12 to modulate gene expression (24). To identify potential regulatory element(s), the 5′ UTR (309 bp) of cobA operon was used to perform the comparative analysis. Conserved-secondary structure and sequence homology analyses demonstrated the presence of a potential VB12 riboswitch (Rfam accession, RF00174, 140 bp), designated as cbiMCbl riboswitch, which contains three major stem-loops (SL), including SL1, SL2, and SL3 (FIG. 5A and FIG. 6C). Mechanistically, SL domains interact with ligands and the targeting RNA to regulate gene expression within various microorganisms (33). Thus, to determine the regulatory function of these SL, the effect of each SL domain on the expression of cbiM and FbFP was tested. Here, the FbFP reporter assays demonstrated SL1-deleted riboswitch (ΔSL1) lost the VB12 dose-dependent regulation, and the IC₅₀ of VB12 was 12962 ng/ml, which was 256 times higher than that of the WT riboswitch (FIGS. 5B and 5C). Consistently, no repression of cbiM expression was observed for ΔSL1 compared to WT riboswitch (FIG. 5D). Further, to precisely elucidate the sites or regions required for VB12-mediated gene regulation, site-directed mutations were introduced into SL1 of the cbiMCbl riboswitch. While WT riboswitch exhibited ˜10-fold repression of FbFP expression at 250 ng/ml VB12, site-mutation of 12 or 13 in SL1 only retained marginal reduction of protein expression even at 2500 ng/ml VB12 (FIG. 5E and FIG. 5F). In contrast, site mutations of 5-6, 7-8 or 9-10 did not impact the regulatory activity of this riboswitch (FIG. 5E and FIG. 5F). Furthermore, null mutation of SL3 resulted in a total loss of gene expression (FIGS. 6A and 6B), whereas deletion of the loop-region of SL3, sites 12′-15′, abolished the dose-dependent downregulation of the downstream gene expression (FIG. 5G and FIG. 5H). In contrast, site-mutation of the loop region did not affect the downregulation of the downstream gene expression by VB12 (FIG. 5G and FIG. 5H), indicating that these sites may cooperatively maintain the regulatory activity of SL3 domain. These findings emphasized that the VB12-mediated regulation was highly dependent on the structure of the cbiMCbl riboswitch.

### Example 5—Translational Regulation of cbiM Expression by RBS-Mediated Base Pairing

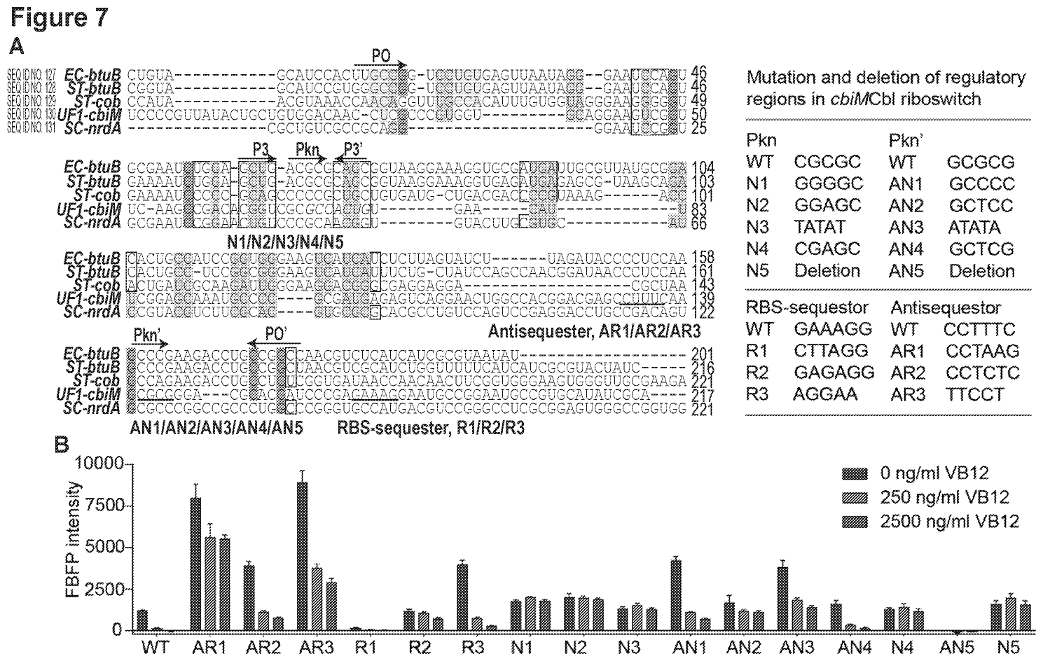

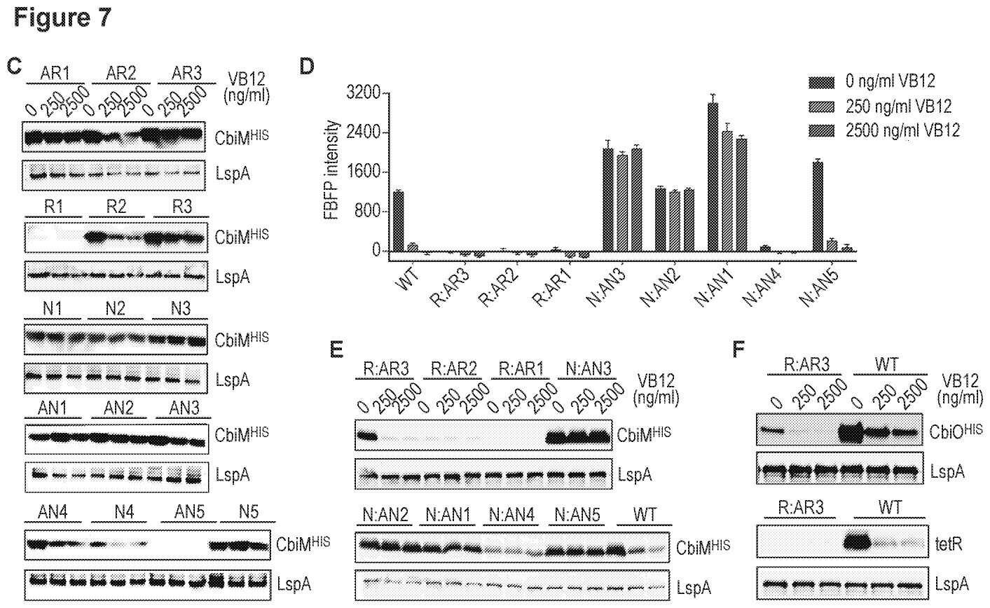

VB12-element exhibits a conserved RNA regulatory sequence in many VB12 riboswitches of numerous microorganisms (15, 29). To investigate whether VB12-element exists within cbiMCbl riboswitch, the known VB12 riboswitches and cbiMCbl riboswitch were compared by sequence alignment analysis using LocARNA, whereby a conserved VB12-element and various secondary structures (e.g., Pkn) were identified within cbiMCbl riboswitch (FIG. 7A). cbiMCbl riboswitch contained a conserved core region, 5′-GCCACUG-3′, which partially overlapped with SL2 domain (FIG. 7A). Importantly, two groups of Watson-Crick base pairs, Pkn:Pkn′ and antisequester:ribosome binding site (RBS)-sequester, were found within cbiMCbl riboswitch (FIG. 7A). To elucidate whether these base pairs are important for VB12-mediated regulatory activity, site-mutation and region-deletion were introduced into these sequences and their impacts on the cbiM and FbFP expression were analyzed (FIG. 7A). Here, single-site mutation (e.g., AR2) or multi-site mutation (e.g., AR3) in the antisequester and RBS-sequester distinctly weakened the regulatory activity of cbiMCbl riboswitch, along with the increased levels of FbFP expression compared to WT riboswitch (FIG. 7B). Further, the regulatory activity of cbiMCbl riboswitch was totally abolished by mutated AR1 or R2 (FIG. 7B). Additionally, the multi-site mutation R1 led to a complete loss of FbFP expression (FIG. 7B). Similarly, disruption of Pkn and Pkn′ base paring differentially impacted the regulatory functions of the riboswitch. For example, the site mutations, N1, N2, N3, N4, N5 and AN2, rendered the mutated riboswitches unable to downregulate FbFP expression (FIG. 7B). While mutations, AN1 and AN3, decreased the regulatory activities of the riboswitches, deletion of Pkn′ (AN5) resulted in complete loss of the regulation and FbFP expression (FIG. 7B). Further, site-mutation of Pkn (AN4) demonstrated comparable regulatory activity to WT riboswitch (FIG. 7B). Additionally, cbiM expression assays were largely consistent with FbFP reporter assays in these riboswitch mutants (FIG. 7B and FIG. 7C). However, R3 and N4 mutants demonstrated the VB12 dose-dependent downregulation of cbiM expression but not of FbFP expression (FIG. 7B and FIG. 7C). Taken together, these data demonstrated that Pkn, Pkn′, antisequester and RBS-sequester were the key regulatory domains of cbiMCbl riboswitch.

Thus far, it is unclear if the regulatory activity of cbiMCbl riboswitch would be dependent on Watson-Crick base pairing between the regulatory domains (Pkn and antisequester) and their complementary domains (Pkn′ and RBS-sequester). Thus, to shed light on this notion, we introduced a second mutation into these single mutants to rescue their base pairing (FIG. 7A). Here, most of the double mutants were still unable to regulate cbiM and FbFP expression (FIGS. 7D and 7E). Interestingly, the double mutant, CbiM-R:AR3-ΔcobA strain, regained the VB12 dose-dependent regulation of cbiM expression but not FbFP expression (FIG. 7D and FIG. 7E, Table 1). These data led to the hypothesis that the base pair-mediated regulation may be influenced by the gene-specific signals. Thus, the expression of cbiO and tetR genes were further analyzed using R:AR3 double mutant (FIG. 5F). Obtained data consistently demonstrated the regulatory activity for cbiO rather than exogenous tetR (FIG. 7F), indicating that cbiMCbl riboswitch specifically regulated the expression of cobA operon by the RBS-mediated base pairing. Together, these data suggested that RBS-mediated Watson-Crick base pairing, as a pivotal factor, facilitated the translational regulation of cobA operon through cbiMCbl riboswitch.

### Example 6—Regulation of cobA Operon Transcription and VB12 Biosynthesis by cbiMCbl Riboswitch

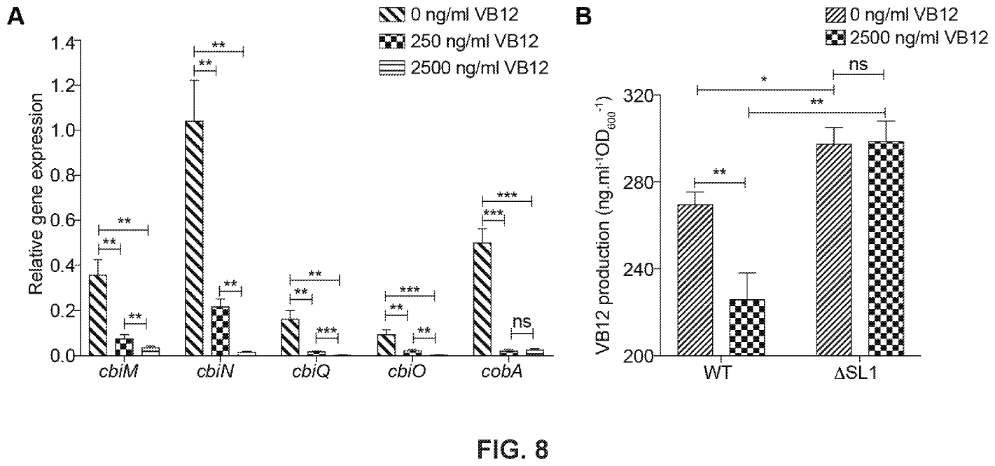

Having determined that VB12 riboswitch controls gene expression through transcriptional and/or translational modifications (24, 34), the impact of the cbiMCbl riboswitch on the transcription of cobA operon was further assessed by qRT-PCR. Obtained data revealed that the mRNA levels of most of the genes, including cbiM, cbiN, cbiO and cbiQ, were significantly repressed in a VB12-dependent manner within P. UF1 (FIG. 8A). To further investigate whether cbiMCbl riboswitch regulates VB12 biosynthesis within P. UF1, the cobA operon was overexpressed under WT and ΔSL1 riboswitches within OW-operon and OΔSL1-operon strains (Table 1). Obtained data demonstrated that disruption of SL1 significantly elevated VB12 production, and this difference was continuously extended in the presence of exogenous VB12 (FIG. 8B), while this effect was abrogated due to SL1 deficiency in this bacterium (FIG. 8B). Altogether, these data demonstrated the cbiMCbl riboswitch negatively controlled the bacterial VB12 biosynthesis through transcriptional and translational gene suppression.

Discussion

Mechanisms involved in bacterial molecular machinery are critically directing the metabolic circuits that regulate the bacterial homeostasis and their stable abundancy, all of which significantly contribute to human health (3, 35-37). One of these metabolites that is biosynthesized by only a few gut bacteria is VB12, which crucially impacts the cross talk between gut microbes and the host (3, 38). Although previous data demonstrated how pathogens, including Salmonella (30, 39), regulate the biosynthesis of this vitamin, the control of VB12 biosynthesis in bacteria with bifidogenic properties, particularly probiotic bacteria, is currently obscure. Thus, to shed light on the mechanistic complexes regulating VB12 in the newly discovered P. UF1 bacterium that controls the local and peripheral immune homeostasis in steady state and during gut infection, we first focused on the genomic region of bacterial CobA. CobA catalyzes the S-adenosyl-L-methionine (SAM)-dependent bismethylation of uroporphyrinogen III synthase (40) to form precorrin-2, the primary precursor of VB12 (18). Thus, deleting cobA from the bacterial chromosome fully abolished VB12 production in ΔcobA P. UF1 strain. Furthermore, VB12 was restored by complementing ΔcobA P. UF1 with cobA gene, illuminating the critical role of cobA in bacterial VB12 synthesis. Further, to elucidate the significance of cobA in VB12 production and its involvement in the regulation of bacterial metabolomic pathways, particularly propionate synthesis, RNA-seq analysis (25) demonstrated cobA deficiency indeed critically altered the transcriptome of P. UF1 whereby metabolic pathways, including porphyrin, chlorophyll, propionate, cysteine, methionine and sulfur metabolisms, were decisively impacted in this bacterium. This was also confirmed through metabolomic analysis of P. UF1 and ΔcobA P. UF1, whereupon various cellular metabolisms, particularly propionate, were impaired, once again highlighting the relevance of cobA in controlling VB12 molecular events that in turn influence the bacterial metabolic homeostasis.

Further, the Examples provided herein also demonstrate that endogenous and exogenous VB12 tightly controlled the expression of cobA operon via its 5′ UTR within P. UF1 bacterium. As shown herein, VB12 completely inhibited the expression of downstream genes by a novel cbiMCbl riboswitch at 750 μM, which was significantly higher than env8HyCbl in E. coli (34).

VB12 analogues displayed the similar regulatory activities in the riboswitch of P. UF1 but not in E. coli (33), possibly as a result of diverse sequences and secondary structures of the VB12 riboswitch. Here, the structure-based analyses revealed that SL1 and SL3 were highly required for the regulatory function of cbiMCbl riboswitch, which belong to receptor domains conserved within the VB12 riboswitches from E. coli and S. typhimurium (29, 33, 41). Further, we clearly demonstrated that Pkn in SL2 and the complementary Pkn′ were crucial regulatory domains within the cbiMCbl riboswitch, whose regulatory function was notably not dependent on Watson-Crick base pairing between the two domains. In contrast, the regulation of env8HyCbl highly depends on the Watson-Crick base pairing of “kissing loop” between SL2 and RBS region (33, 34), indicating cbiMCbl riboswitch within P. UF1 may employ a new molecular mechanism to control gene expression. Interestingly, a novel Watson-Crick base pairing between the RBS-sequester and antisequester was identified in the Examples herein, whose base paring was essentially required for regulatory activities of the riboswitch. In E. coli, it has been well documented that the env8HyCbl riboswitch regulates gene expression at transcriptional and translational levels (33, 34), as found for cbiMCbl riboswitch. In Listeria monocytogenes and S. typhimurium, VB12 riboswitches don't control the transcription of the downstream genes (24, 41), indicating that various VB12 riboswitches may display a distinct regulatory model for gene expression in bacteria with different natures and functions, mainly pathogenic, or beneficial bacteria such probiotics.

The probiotics are defined as live microbial feed supplements with beneficial properties, which potentially benefit the host when administered in adequate amounts (42). Propionibacterium species are currently of great interest for their beneficial effects as probiotics and are applied to human dietary consumption, including Swiss cheese (43). Here, we demonstrated that cbiMCbl riboswitch controlled the expression of the cobA operon at transcriptional and translational levels within this bacterium and that the genetic modification of this riboswitch significantly enhances VB12 biosynthesis within P. UF1. Henceforth, it is reasonable to speculate that this bacterium abundantly synthesizing VB12 may critically contribute to the observed immune homeostasis in the host.

VB12 can be used by more than 80% of gut microbiota (3), suggesting that this vitamin can be utilized to support the healthy ecology of the gut microbiota involved in the intestinal immune homeostasis of the host (3, 44). Here, we clearly demonstrated how precisely VB12 biosynthesis was regulated within cobA through a new riboswitch, cbiMCbl, within P. UF1. This riboswitch may serve as a novel target to increase the levels of VB12 by ingesting a probiotic bacterium with bifidogenic properties that can contribute to enhanced cross-feeding of neighboring gut bacteria, that potentially mitigate the symptoms of intestinal disorders in the near future (45).

### REFERENCES CITED

1. Crofts et al., Chemistry & Biology 20:1265-1274, 2013.
2. Fang et al., Microb Cell Fact 16:15, 2017.
3. Degnan et al., Cell Metabolism 20:769-778, 2014.
4. Obeid et al., Mol Nutr Food Res 59:1364-72, 2015.
5. Roth et al., Annu Rev Microbiol 50:137-81, 1996.
6. Fenech et al., Mutat Res 475:1-6, 2001.
7. Fenech M., Mutat Res 475:57-67, 2001.
8. Fenech et al., Carcinogenesis 19:1163-71, 1998.
9. Dror et al., Nutr Rev 66:250-5, 2008.
10. Clarke et al., Br J Nutr 100:1054-9, 2008.
11. Mills et al., Birth Defects Research Part a-Clinical and Molecular Teratology 82:636-643, 2008.
12. Molloy et al., Food Nutr Bull 29:S101-11; discussion S112-5, 2008.
13. Kvestad et al., American Journal of Clinical Nutrition 105:1122-1131, 2017.
14. O'Leary et al., Nutrients 2:299-316, 2010.
15. Rodionov et al., J Biol Chem 278:41148-59, 2003.
16. Warren et al., Nat Prod Rep 19:390-412, 2002.
17. Piwowarek et al., Applied Microbiology and Biotechnology 102:515-538, 2018.
18. Sattler et al., Journal of Bacteriology 177:1564-1569, 1995.
19. Escalantesemerena et al., Journal of Bacteriology 172:273-280, 1990.
20. Falentin et el., Plos One 5, 2010.
21. Cousin et al., Food Microbiology 32:135-146, 2012.
22. Vitreschak et al., RNA 9:1084-97, 2003.
23. Barrick et al., Genome Biol 8:R239, 2007.
24. Mellin et al., Science 345:940-943, 2014.
25. Colliou et al., Journal of Clinical Investigation 127:3970-3986, 2017.
26. Colliou et al., Gut Microbes 9:279-287, 2018.
27. Ge Y et al., P. UF1. Mucosal Immunology 12:434-444, 2019.
28. Black MM., Food Nutr Bull 29:S126-31, 2008.
29. Nahvi et al., Nucleic Acids Res 32:143-50, 2004.
30. Richter-Dahlfors et al., Mol Microbiol 13:541-53, 1994.
31. Lundrigan et al., Proc Natl Acad Sci USA 88:1479-83, 1991.
32. Lobo L A, et al., Microbiol Lett 317:67-74, 2011.
33. Johnson et al., Nature 492:133, 2012.
34. Polaski et al., Cell Reports 15:1100-1110, 2016.
35. Dantas et al., Annu Rev Microbiol 67:459-75, 2013.
36. Diaz et al., Proc Natl Acad Sci USA 108:3047-52, 2011.
37. Goodman et al., Cell Metabolism 12:111-116, 2010.
38. Lupski et al., Am J Hum Genet 58:21-7, 1996.
39. Wu et al., J Bacteriol 174:4833-7, 1992.
40. Mathews et al., EMBO J 20:5832-9, 2001.
41. Ravnum et al., Mol Microbiol 23:35-42, 1997.
42. Hill et al., Nature Reviews Gastroenterology & Hepatology 11:506-514, 2014.
43. Angelopoulou et al., International Dairy Journal 66:135-139, 2017.
44. Cordonnier et al., Toxins (Basel) 8, 2016.
45. McNulty et al., Sci Transl Med 3:106ra106, 2011.
46. Kosmider et al., Bioresour Technol 105:128-33, 2012.
47. Li et al., PLoS Comput Biol 9:e1003123, 2013.
48. Teng et al., Microbiol Methods 119:37-43, 2015.

What is claimed is:

1. A Propionibacterium that has been modified to overproduce vitamin B12.

2. The bacterium of claim 1, that is P. freudenreichii.

3. The bacterium of claim 1, that is P. UF1.

4. The bacterium of any one of claims 1-3, comprising a mutation in a vitamin B12 riboswitch that decreases activity of the vitamin B12 riboswitch.

5. The bacterium of claim 4, wherein the vitamin B12 riboswitch is cbiMCbl.

6. The bacterium of claim 4 or claim 5, wherein the mutation is deletion of stem loop 1 (SL1) of the cbiMCbl riboswitch.

7. The bacterium of any one of claims 4-6, wherein the vitamin B12 riboswitch retains stem loops 2 and 3.

8. A composition comprising the bacterium of any one of claims 1-7.

9. The composition of claim 8, wherein the composition is a food product.

10. The composition of claim 9, wherein the composition is a beverage.

11. A method of increasing vitamin B12 production in a bacterium, the method comprising:
(a) providing a bacterium comprising a vitamin B12 riboswitch; and
(b) introducing a mutation in a vitamin B12 riboswitch that decreases activity of the vitamin B12 riboswitch, thereby increasing vitamin B12 production.

12. The method of claim 11, wherein the bacterium is Pseudomonas dentrificans, Rhodobacter capusulatus, Rhodobacter sphaeroides, Sinorhizobium meliloti, Salmonella typhimurium, Bacillus megaterium, Propionibacterium shermanii, Escherichia coli, Thermotoga sp. RQ2, Thermotoga maritima MSB8, Thermotoga neapolitana, Thermotoga petrophila, Thermotoga naphthophila, Thermotoga thermarum, Thermotoga lettingae, Fervidobacterium nodosum, Thermosipho melanesiensis, Thermosipho africanus, Kosmotoga olearia, Mesotoga prima or Petrotoga mobilis.

13. The method of claim 11, wherein the bacterium is Pseudomonas denitrificans, Propionibacterium shermanii, Sinorhizobium meliloti or Escherichia coli.

14. The method of claim 11, wherein the bacterium is Propionibacterium.

15. The method of claim 11, wherein the bacterium is P. freudenreichii.

16. The method of claim 11, wherein the bacterium is P. UF1.

17. The method of claim 11, wherein the vitamin B12 riboswitch is cbiMCbl.

18. The method of claim 11, wherein the mutation is deletion of stem loop 1 (SL1) of the cbiMCbl riboswitch.

18. The method of claim 11, further comprising culturing the bacterium under conditions sufficient to produce vitamin B12.

19. The method of any one of claims 11-18, further comprising isolating the vitamin B12 produced from the bacterium.

20. A method of producing vitamin B12, the method comprising
(a) culturing the bacterium of claim 1 under conditions sufficient to produce vitamin B12; and
(b) isolating vitamin B12 from the bacterium.

21. The method of claim 20, wherein the bacterium is Propionibacterium.

22. The method of claim 20, wherein the bacterium is P. freudenreichii.

23. The method of claim 20, wherein the bacterium is P. UF1.

24. A method of increasing vitamin B12 production in the gut of a mammalian subject comprising administering to the subject a composition of any one of claims 8-10.

25. A method of increasing vitamin B12 production in the gut of a mammalian subject comprising administering to the subject a composition comprising a Propionibacterium and an agent that inhibits the activity of a vitamin B12 riboswitch in the Propionibacterium.

26. The method of claim 25, wherein the vitamin B12 riboswitch is cbiMCbl.

27. A method of increasing vitamin B12 production in the gut of a mammalian subject comprising administering to the subject a composition comprising a bacterium comprising a vitamin B12 riboswitch; and an agent that inhibits the activity of the vitamin B12 riboswitch in the bacterium.

28. The method of claim 27, wherein the bacterium is Pseudomonas dentrificans, Rhodobacter capusulatus, Rhodobacter sphaeroides, Sinorhizobium meliloti, Salmonella typhimurium, Bacillus megaterium, Propionibacterium shermanii, Escherichia coli, Thermotoga sp. RQ2, Thermotoga maritima MSB8, Thermotoga neapolitana, Thermotoga petrophila, Thermotoga naphthophila, Thermotoga thermarum, Thermotoga lettingae, Fervidobacterium nodosum, Thermosipho melanesiensis, Thermosipho africanus, Kosmotoga olearia, Mesotoga prima or Petrotoga mobilis.
